(19) United States
(12) Patent Application Publication
(10) Pub. No.: US 2021/0338215 A1
(43) Pub. Date: Nov. 4, 2021

(54) TISSUE RESECTION SYSTEMS AND METHODS FOR DIAGNOSTIC AND THERAPEUTIC IMPLEMENTATION

(71) Applicant: Ethicon, Inc., Somerville, NJ (US)

(72) Inventors: William Cohn, Houston, TX (US); Terry Daglow, Houston, TX (US); Matthew Kuhn, Houston, TX (US); Sinead Emily Miller, Houston, TX (US); Steven Nguyen, Cyprus, TX (US)

(21) Appl. No.: 17/226,650

(22) Filed: Apr. 9, 2021

Related U.S. Application Data
(60) Provisional application No. 63/017,741, filed on Apr. 30, 2020.

(51) Int. Cl. A61B 10/04; A61B 10/02; A61B 34/30; A61B 34/20; A61K 9/00; A61K 33/243

(52) U.S. Cl. CPC A61B 10/04; A61B 10/0266; A61B 34/30; A61B 34/20; A61K 9/0024; A61B 2090/3762; A61B 2010/045; A61B 2034/303; A61B 2034/2051; A61B 2034/2063; A61K 33/243

(57) ABSTRACT

A method for removing tissues may comprise disposing a tissue resection device at a target tissue site, causing the tissue resection device to resect a core of tissue from the target tissue site, removing the core of tissue from the body, wherein the removing the core of tissue from the body creates a core cavity at the target tissue site.

### CROSS-REFERENCE TO RELATED APPLICATIONS

This application claims priority to and the benefit of United States U.S. Patent Application No. 63/017,741 filed Apr. 30, 2020, which is hereby incorporated by reference in their entirety.

### BACKGROUND

In certain instances, tissue may need to be removed from the body. As an example, cancerous or infected tissue may be removed from the body as part of a treatment. Cancer is not a single disease, but rather a collection of related diseases that may start essentially anywhere in the body. Common amongst all types of cancer is that the body's cells begin to divide without stopping, proliferating and potentially spreading into surrounding tissues. In the normal course of events, cells grow and divide to form new cells as required by the body and when they become damaged or old, they die, and new cells replace the damaged or old cells; however, cancer interrupts this process. With cancer, the cells become abnormal, and cells that should die do not and new cells form when they are not needed. These new cells may reproduce or proliferate without stopping and may form growths called tumors.

Cancerous tumors are malignant, which means they may spread into or invade surrounding healthy tissue. In addition, cancer cells may break off and travel to remote areas in the body through blood or in the lymph system. Benign tumors, unlike malignant tumors, do not spread or invade surrounding tissue; however, they may grow large and cause damage. Both malignant and benign tumors may be removed or treated. Malignant tumors tend to grow back whereas benign tumors may grow back but are much less likely to do so.

Cancer is a genetic disease in that it is caused by changes in the genes that control the ways that cells function, especially in how they grow and divide. Genetic changes that cause cancer may be inherited or they may arise over an individual's lifetime as a result of errors that occur as cells divide or because of damage to DNA caused by certain environmental exposure, for example, industrial/commercial chemicals and ultraviolet light. The genetic changes that may cause cancer tend to affect three types of genes; namely proto-oncogenes which are involved in normal cell growth and division, tumor suppressor genes which are also involved in controlling cell growth and division, and DNA repair genes which, as the name implies, are involved in repairing damaged DNA.

More than one-hundred distinct types of cancer have been identified. The type of cancer may be named for the organ or tissue where the cancers arise, for example, lung cancer, or the type of cell that formed them, for example squamous cell cancer. Cancer, unfortunately, is a leading cause of death both in the United States and world-wide. According to the World Health Organization, the number of new cancer cases will rise to twenty-five (25) million per year over the next two decades.

Lung cancer is one of the most common cancers today. According to the World Cancer Report 2014 from the World Health Organization, lung cancer occurred in 14 million people and resulted in 8.8 million deaths world-wide, making it the most common cause of cancer-related death in men and the second most common cause of cancer-related death in women. Lung cancer or lung carcinoma is a malignant lung tumor that if left untreated may metastasize into neighboring tissues and organs. The majority of lung cancer is caused by long-term tobacco smoking; however, about 10 to 15 percent of lung cancer cases are not tobacco related. These non-tobacco cases are most often caused by a combination of genetic factors and exposure to certain environmental conditions, including radon gas, asbestos, second-hand tobacco smoke, other forms of air pollution, and other agents. The chance of surviving lung cancer as well as other forms of cancer depends on early detection and treatment.

Improvements in removing tissue are needed.

### SUMMARY

It may be desirable to remove a core of tissue from other target tissue sites including, but not limited to, the lungs, the liver, pancreas, or gastrointestinal (GI) tract, for which managing post-coring bleeding may be desired. A core of tissue may have a prescribed (e.g., pre-defined) shape (e.g., columnar) and dimension based on a coring apparatus. Such coring apparatus may be used to core the same or substantially the same shaped tissue core in a repeatable manner. Such coring may be distinguished from other tissue removal, for example using scissors or scalpel, where the cut tissue will not have a pre-defined shape or dimensions.

Methods may comprise removing a core of tissue from a tissue site. Such coring may further comprise introducing a tissue resection device to a tissue site, using the tissue resection device to create a core of tissue, removing the core of tissue from the body to create a tissue cavity, and sealing the tissue cavity.

In certain aspect, removing a core of tissue from a tissue site may further comprise one or more of: determining the location of a tissue lesion using one or more imaging modalities, navigating an instrument to the tissue site such as the tissue lesion (with and without image guidance), coupling (e.g., anchoring) the instrument to the tissue lesion, obtaining access to the tissue site (making an incision, introduction through a port/trocar, or direct access via an open procedure), introducing a tissue resection device to the tissue site (with and without using the anchor as a guide), using the tissue resection device to create a core of tissue or amputating the core of tissue from the tissue site, removing the core of tissue from the body (with and without leaving a cavity “access sleeve”), analyzing the tissue core sample (tissue histology, ROSE, DNA sequencing, etc.), sealing the tissue cavity, removing some or all instrumentation, or closing tissue access points.

In certain aspects, removing a core of tissue from a tissue site and subsequent diagnosis may further comprise one or more of: determining a location of a tissue lesion using one or more imaging modalities, navigating an instrument to a tissue site such as the tissue lesion (with and without image guidance), coupling (e.g., anchoring) the instrument to the tissue lesion, obtaining access to the tissue site (making an incision, introduction through a port/trocar, or direct access via an open procedure), introducing a tissue resection device to the tissue site (with and without using the anchor as a guide), using the tissue resection device to create a core of tissue or amputating the core of tissue from the tissue site, removing the core of tissue from the body (with and without leaving a cavity “access sleeve”), analyzing the tissue core sample (tissue histology, ROSE, DNA sequencing, etc.), sealing the tissue cavity, removing some or all instrumentation, or closing tissue access points.

In certain aspects, removing a core of tissue from a tissue site, subsequent diagnosis, and therapeutic management of confirmed malignancy may further comprise one or more of: determining the location of a tissue lesion using one or more imaging modalities, navigating an instrument to the tissue lesion (with and without image guidance), coupling (e.g., anchoring) the instrument to the tissue lesion, obtaining access to the tissue site (making an incision, introduction through a port/trocar, or direct access via an open procedure), introducing a tissue resection device to the tissue site (with and without using the anchor as a guide), using the tissue resection device to create a core of tissue or amputating the core of tissue from the tissue site, removing the core of tissue from the body (with and without leaving a cavity “access sleeve”), analyzing the tissue core sample (tissue histology, ROSE, DNA sequencing, etc.), performing therapeutic management of tissue such as benign or malignant tissue, sealing the tissue cavity, removing some or all instrumentation, closing tissue access points.

Methods for coring tissue may comprise disposing a tissue resection device at a target tissue site, causing the tissue resection device to resect a core of tissue from the target tissue site, and removing the core of tissue from the body, wherein the removing the core of tissue from the body creates a core cavity at the target tissue site. The core of tissue comprises at least a portion of a tissue lesion. The resecting the core of tissue from the target tissue site may comprise mechanical transection. The resecting the core of tissue from the target tissue site may comprise the delivery of radiofrequency energy. The resecting the core of tissue from the target tissue site may comprise mechanical compression and the delivery of radiofrequency energy. The resecting the core of tissue from the target tissue site may comprise transection with an energized wire. The resecting the core of tissue from the target tissue site may comprise one of more of mechanical compression, the delivery of radiofrequency energy, the delivery of microwave energy, the delivery of ultrasonic energy, or transection with an energized wire. Other resection devices and procedures may be used. The resection device may be configured for one or more of mechanical compression, the delivery of radiofrequency energy, the delivery of microwave energy, the delivery of ultrasonic energy, or transection with an energized wire.

Methods for coring tissue may further comprise inserting a sleeve into the core cavity to support a wall of the core cavity. Methods for coring tissue may further comprise delivering radiofrequency energy to at least a portion of a wall defining the core cavity. Methods for coring tissue may further comprise delivering chemotherapy to at least a portion of a wall defining the core cavity. Methods for coring tissue may further comprise delivering microwave energy to at least a portion of a wall defining the core cavity. Methods for coring tissue may further comprise delivering thermal energy to at least a portion of a wall defining the core cavity. Methods for coring tissue may further comprise delivering ultrasonic energy to at least a portion of a wall defining the core cavity.

Methods for coring tissue may further comprise sealing biological fluid vessels. The sealing biological fluid vessels may minimize flow of biological fluids into the cavity core. The sealing may be effected using at least mechanical compression. The sealing may be effected using at least radiofrequency energy. The sealing may be effected using at least microwave energy. The sealing may be effected using at least ultrasonic energy. The sealing may be effected using one or more of compression or delivery of energy such as radiofrequency, microwave, ultrasonic, or thermal energy.

The present disclosure relates to a system, device and method for performing lung lesion removal. A lung needle biopsy is typically performed when an abnormality is found on an imaging test, for example, an X-ray or CAT scan. In a lung needle biopsy, a fine needle is used to remove sample of lung tissue for examining under a microscope to determine the presence of abnormal cells. Tissue diagnosis is challenging in small (<6 mm) and intermediate (6-12 mm) nodules. CT guided biopsy of peripheral lesions, either through the chest wall (80%) or by means of a bronchoscope (20%) yields only a 0.001-.002 cm2 of diagnostic tissue, and as such, cancer, when present, is only successfully identified in 60% of small and intermediate nodules. Although bronchoscopic techniques and technology continue to evolve, biopsy accuracy, specificity, and sensitivity will always be limited when dealing with small and intermediate nodules in the periphery of the lung.

If it is determined that the lesion is cancerous, a second procedure may be performed to remove the lesion and then followed up with chemotherapy and/or radiation. The second procedure most likely involves lung surgery. These procedures are typically done through an incision between the ribs. There are a number of possible procedures depending on the state of the cancer. Video-assisted thoracic surgery is a less invasive procedure for certain types of lung cancer. It is performed through small incisions utilizing an endoscopic approach and is typically utilized for performing wedge resections of smaller lesions close to the surface of a lung. In a wedge resection, a portion of the lobe is removed. In a sleeve resection, a portion of a large airway is removed thereby preserving more lung function.

Nodules deeper than 2-3 cm from the lung surface, once identified as suspicious for cancer, are difficult to localize and excise using laparoscopic or robotic lung sparing technique despite pre-procedure image guided biopsy and localization. Thus, surgeons perform an open thoracotomy or lobectomy to remove lung nodules that are 2-3 cm from the lung surface. A thoracotomy is an open approach surgery in which a portion of a lobe, a full lobe or an entire lung is removed. In a pneumonectomy, an entire lung is removed. This type of surgery is obviously the most aggressive. In a lobectomy, an entire section or lobe of a lung is removed and represents a less aggressive approach than removing the entire lung. All thoracoscopic lung surgeries require trained and experienced thoracic surgeons and the favorability of surgical outcomes track with operative experience.

Any of these types of lung surgery is a major operation with possible complications which depend on the extent of the surgery as well as the patient's overall health. In addition to the reduction in lung function associated with any of these procedures, the recovery may take from weeks to months. With a thoracotomy, spreading of the ribs is required, thereby increasing postoperative pain. Although video-assisted thoracic surgery is less invasive, there may still be a substantial recovery period. In addition, once the surgery is complete, full treatment may require a system chemotherapy and/or radiation treatment.

As set forth above, a fine needle biopsy may not prove to be totally diagnostic. The fine needle biopsy procedure involves guiding a needle in three-dimensional space under two-dimensional imaging. Accordingly, the doctor may miss the lesion, or even if he or she hits the correct target, the section of the lesion that is removed through the needle may not contain the cancerous cells or the cells necessary to assess the aggressiveness of the tumor. A needle biopsy removes enough tissue to create a smear on a slide. The device of the present disclosure is designed to remove the entire lesion, or a substantial portion of it, while minimizing the amount of healthy lung tissue removal. This offers a number of advantages. Firstly, the entire lesion may be examined for a more accurate diagnosis without confounding sampling error, loss of cell packing or gross architecture. Secondly, since the entire lesion is removed, a secondary procedure as described above may not be required. Thirdly, localized chemotherapy and/or energy-based tumor extirpation, such as radiation, may be introduced via the cavity created by the lesion removal.

In at least one embodiment, the disclosure defines a method for removing a tissue lesion including coupling (e.g., anchoring) to the tissue lesion; creating a channel in the tissue leading to the tissue lesion; creating a tissue core including the tissue lesion; ligating the tissue core at a ligation point downstream from the tissue lesion; amputating the tissue core form the tissue between the ligation point and the tissue lesion; and removing the tissue core from the channel.

In keeping with aspects of the disclosure, the sleeve may be inserted in the channel prior to or after removing the tissue core. The sleeve may also be anchored to the tissue. In keeping with another aspect of the disclosure, a localized treatment may be delivered through the sleeve.

In some embodiments, creating a tissue core includes cauterizing and cutting tissue. Ligating tissue may include tissue may include cauterizing tissue at a specific location known as the ligation point. Amputation of the tissue core may be performed with a snare, an energized wire or any other device capable of slicing tissue.

In some embodiments, the tissue core is created by first sealing blood vessels then slicing tissue to form the core.

### BRIEF DESCRIPTION OF THE DRAWINGS

The following drawings show generally, by way of example, but not by way of limitation, various examples discussed in the present disclosure. In the drawings:

FIG. 1 shows an example method in accordance with the present disclosure.

FIG. 2 shows an example method in accordance with the present disclosure.

FIG. 3 shows an example method in accordance with the present disclosure.

FIG. 4 shows an example method in accordance with the present disclosure.

FIG. 5 illustrates a blade with an open channel.

FIG. 6 illustrates a distal tip of the blade of FIG. 5.

FIG. 7 illustrates a distal end of air channel connected to a flexible or rigid tube.

FIGS. 8A-8B illustrate an example trocar.

FIGS. 9A-9B illustrate an example trocar.

FIG. 10 illustrates an example trocar.

FIG. 11 depicts a tissue resection device in accordance with an embodiment of the present disclosure.

FIG. 12 illustrates a sectional view of the tissue resection device of FIG. 11.

FIG. 13 shows a sectional view of a tissue resection device in accordance with an embodiment of the present disclosure.

FIG. 14 depicts a sectional view of a tissue resection device in accordance with an embodiment of the present disclosure.

FIG. 15 illustrates an exemplary anchor that may be employed in a lesion removal method in accordance with an embodiment of the present disclosure.

FIG. 16 shows a series of incision blades for use in a lesion removal method in accordance with an embodiment of the present disclosure.

FIG. 17 displays tissue dilators suitable for use in a lesion removal method in accordance with an embodiment of the present disclosure.

FIG. 18 shows an example workflow of tissue sample analysis.

FIG. 19 shows an application of an example system for sealing tissue.

FIG. 20 shows an application of an example system for sealing tissue.

FIGS. 21A, 21B, and 21C show an application of an example system for sealing tissue.

FIGS. 22A and 22B show an application of an example system for sealing tissue.

FIGS. 23A, 23B, and 23C show an application of an example system for sealing tissue.

FIG. 24 illustrates an example therapy system and method in accordance with the present disclosure.

FIG. 25 illustrates an example therapy system and method in accordance with the present disclosure.

FIG. 26 illustrates an example therapy system and method in accordance with the present disclosure.

FIG. 27 illustrates an example therapy system and method in accordance with the present disclosure.

FIG. 28 illustrates an example therapy system and method in accordance with the present disclosure.

FIG. 29 illustrates an example therapy system and method in accordance with the present disclosure.

FIG. 30 illustrates an example therapy system and method in accordance with the present disclosure.

### DETAILED DESCRIPTION

The present disclosure relates to systems and methods for coring tissue. Various tissue and sites may benefit from the disclosed systems and methods.

A core of tissue may have a prescribed (e.g., pre-defined) shape (e.g., columnar) and dimension based on a coring apparatus. Such coring apparatus may be used to core the same or substantially the same shaped tissue core in a repeatable manner. Such coring may be distinguished from other tissue removal, for example using scissors or scalpel, where the cut tissue will not have a pre-defined shape or dimensions.

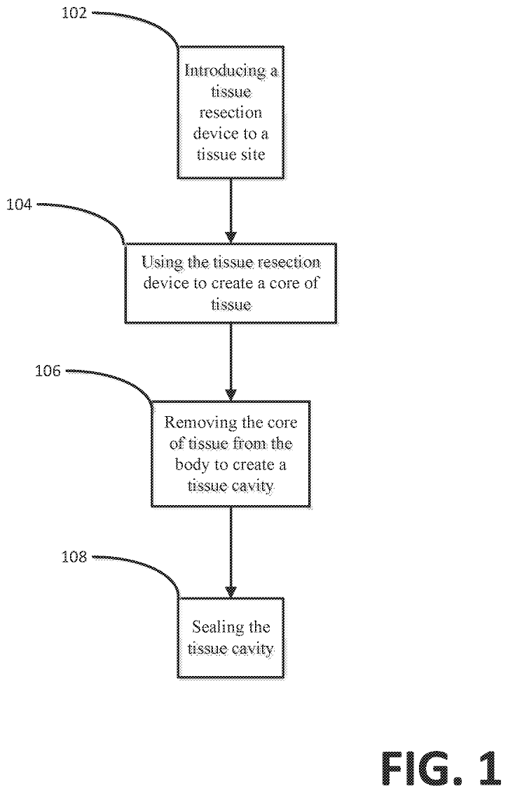

FIG. 1 shows an example method, which may comprise removing a core of tissue from a tissue site. Such coring may further comprise introducing a tissue resection device to a tissue site (102), amputating a core of tissue such as using the tissue resection device to create a core of tissue (104), removing the core of tissue from the body to create a tissue cavity (106), and sealing the tissue cavity (108).

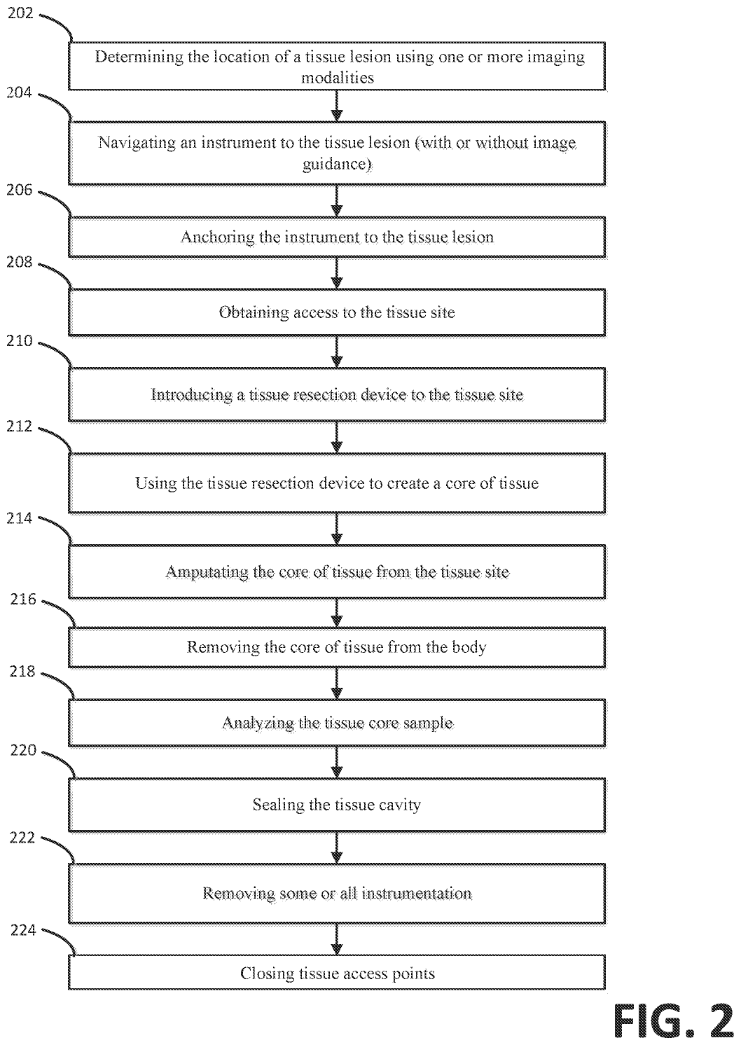

As illustrated in FIG. 2, removing a core of tissue from a tissue site may further comprise one or more of: determining the location of a tissue lesion using one or more imaging modalities (202), navigating an instrument to a site such as the tissue lesion (with and without image guidance) (204), coupling (e.g., anchoring) the instrument to the tissue lesion (206), obtaining access to the tissue site (making an incision, introduction through a port/trocar, or direct access via an open procedure) (208), introducing a tissue resection device to the tissue site (with and without using the anchor as a guide) (210), using the tissue resection device to create a core of tissue (212) or amputating the core of tissue from the tissue site (214), removing the core of tissue from the body (with and without leaving a cavity “access sleeve”) (216), analyzing the tissue core sample (tissue histology, ROSE, DNA sequencing, etc.) (218), sealing the tissue cavity (220), removing some or all instrumentation (222), or closing tissue access points (224).

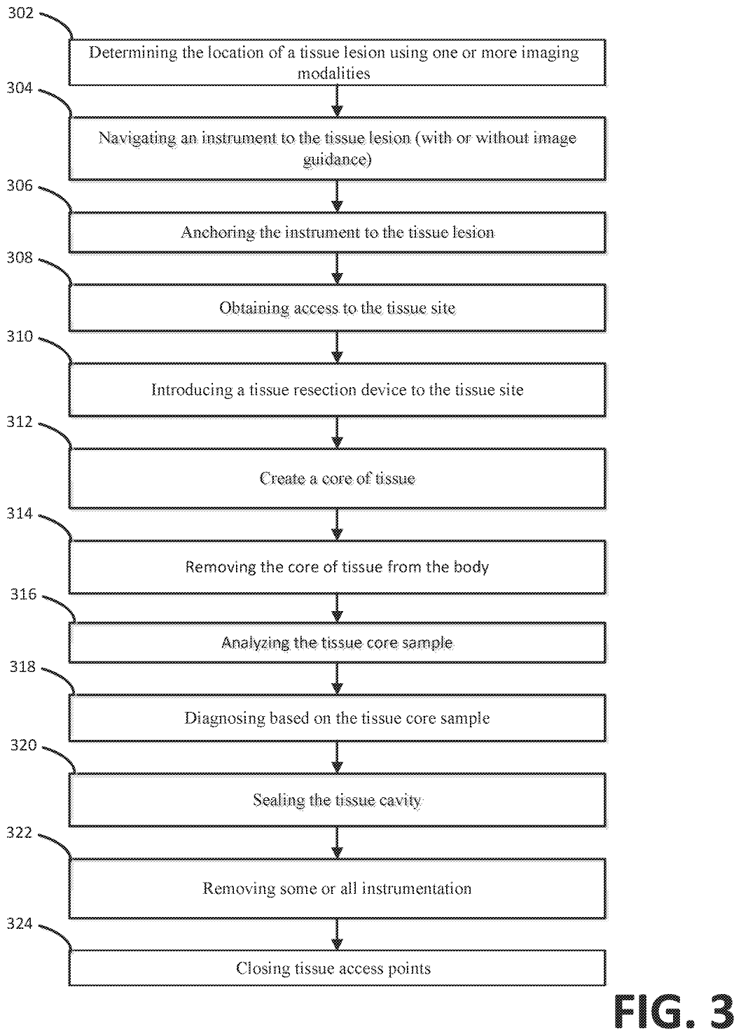

As illustrated in FIG. 3, removing a core of tissue from a tissue site and subsequent diagnosis may further comprise one or more of: determining a location of a tissue lesion using one or more imaging modalities (302), navigating an instrument to a site such as the tissue lesion (with and without image guidance) (304), coupling (e.g., anchoring) the instrument to the tissue lesion (306), obtaining access to the tissue site (making an incision, introduction through a port/trocar, or direct access via an open procedure) (308), introducing a tissue resection device to the tissue site (with and without using the anchor as a guide) (310), using the tissue resection device to create a core of tissue (312) or amputating the core of tissue from the tissue site, removing the core of tissue from the body (with and without leaving a cavity “access sleeve”) (314), analyzing the tissue core sample (tissue histology, ROSE, DNA sequencing, etc.) (316), diagnosing based on at least the tissue core sample (318), sealing the tissue cavity (320), removing some or all instrumentation (322), or closing tissue access points (324).

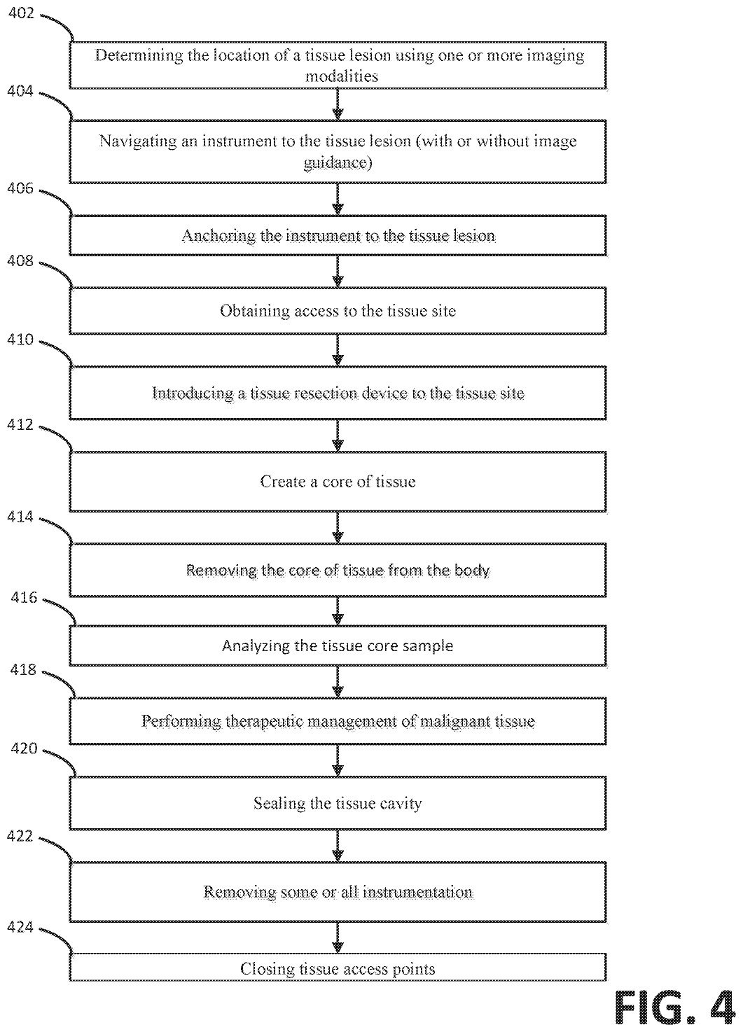

As illustrated in FIG. 4, removing a core of tissue from a tissue site, subsequent diagnosis, and therapeutic management of confirmed malignancy may further comprise one or more of: determining the location of a tissue lesion using one or more imaging modalities (402), navigating an instrument to a site such as the tissue lesion (with and without image guidance) (404), coupling (e.g., anchoring) the instrument to the tissue lesion (406), obtaining access to the tissue site (making an incision, introduction through a port/trocar, or direct access via an open procedure) (408), introducing a tissue resection device to the tissue site (with and without using the anchor as a guide) (410), using the tissue resection device to create a core of tissue or amputating the core of tissue from the tissue site (412), removing the core of tissue from the body (with and without leaving a cavity “access sleeve”) (416), analyzing the tissue core sample (tissue histology, ROSE, DNA sequencing, etc.) (418), performing therapeutic management of tissue such as benign or malignant tissue (418), sealing the tissue cavity (420), removing some or all instrumentation (422), and closing tissue access points (424).

The present disclosure relates to methods and systems for coring tissue. Methods for coring tissue may comprise disposing a tissue resection device at a target tissue site, causing the tissue resection device to resect a core of tissue from the target tissue site, and removing the core of tissue from the body. The removing the core of tissue from the body may create a core cavity at the target tissue site. The core of tissue may comprise at least a portion of a tissue lesion. The resecting the core of tissue from the target tissue site may comprise mechanical transection. The resecting the core of tissue from the target tissue site may comprise the delivery of radiofrequency energy. The resecting the core of tissue from the target tissue site may comprise mechanical compression and the delivery of radiofrequency energy. The resecting the core of tissue from the target tissue site may comprise transection with an energized wire. The resecting the core of tissue from the target tissue site may comprise one of more of mechanical compression, the delivery of radiofrequency energy, the delivery of microwave energy, the delivery of ultrasonic energy, or transection with an energized wire. Other resection devices and procedures may be used. The resection device may be configured for one or more of mechanical compression, the delivery of radiofrequency energy, the delivery of microwave energy, the delivery of ultrasonic energy, or transection with an energized wire.

The present disclosure relates to methods and systems for coring tissue and sealing the core cavity created by removing the tissue core. Such methods may comprise disposing a fill material in the core cavity. Methods may comprise applying pressure to a portion of the core cavity such as to a wall defining the core cavity. Methods may comprise ablating a portion of the core cavity such as a wall defining the core cavity. Methods may comprise causing a cavity closure device, such as suture thread, a stapling device, an ultrasonic tissue sealing device, a bipolar radiofrequency sealing device, or any combination thereof to close the tissue cavity. Methods may comprise disposing a cavity sealing material, such as a tissue graft, a hemostatic patch, a hemostatic agent such as fibrin or thrombin, a biological adhesive material such as Dermabond®, or any combination thereof to close the tissue cavity.

Methods may comprise any combination or permutation of: 1) disposing an anchoring device into a tissue cavity, 2) disposing a tissue access port into the tissue cavity, 3) disposing a tissue sealing device into the tissue cavity (with or without a tissue access port, with or without guidance from an anchoring device), 4) causing the tissue sealing device to seal at least a portion of the tissue cavity, 5) introducing a fill material into the tissue cavity (with or without a fill material delivery device, with or without being proceeded by disposing a tissue sealing device into the tissue cavity, with or without removing the tissue sealing device after sealing at least a portion of the tissue cavity, with or without a tissue access port), 6) disposing a cavity sealing material adjacent to the tissue cavity (with or without being proceeded by disposing a tissue sealing device into the tissue cavity, with or without removing the tissue sealing device after sealing at least a portion of the tissue cavity, with or without being proceeded by introducing a fill material into the tissue cavity), 7) disposing a cavity closure device adjacent to the tissue, and 8) causing a cavity closure device to close the tissue cavity (with or without being proceeded by any combination or permutation of the above steps). As described herein, methods may be used to core and/or seal tissue at various target sites. Although a lung is used as an illustrative example, it should not be so limiting, as other target sites may be punctured or actively cored and may benefit from the disclosed sealing methods.

Imaging Systems

Various systems, devices, and apparatus may be used to locate a target site such as a target tissue site in a human body. For example, imaging systems may be used such as computed tomography (CT), ultrasound, magnetic resonance imaging (MM), endoscope, visual, electromagnetic, and/or X-ray.

CT

In conventional X-ray systems, a beam of X-rays is directed through an object such as the human body onto a flat X-ray photographic film. The beam of X-rays is selectively absorbed by structures within the object, such as bones within the human body. Since the exposure of the X-ray film varies directly with the transmission of X-rays through the body (and varies inversely with the absorption of X-rays), the image that is produced provides an accurate indication of any structures within the object that absorbed the X-rays. As a result, X-rays have been widely used for non-invasive examination of the interior of objects and have been especially useful in the practice of medicine.

The image that is formed from the X-ray is basically the shadow of the structures within the object that absorb the X-rays. As a result, the image formed on the X-ray is only two-dimensional, and if multiple X-ray absorbing structures lie in the same shadow, information about some of these structures is likely to be obscured. Moreover, in the case of medical applications, it is often quite difficult to use conventional X-ray systems to examine portions of the body such as the lungs that consist mostly of air when inflated and do not absorb X-rays significantly.

Many of the limitations of conventional X-ray systems may be avoided by X-ray computer tomography, which is often referred to as CT. In particular, CT provides three-dimensional views and the imaging of structures and features that are unlikely to be seen very well in a conventional X-ray.

A CT scanning equipment typically includes a computer, a large toroidal structure and a platform that is movable along a longitudinal axis through the center of the toroidal structure. Mounted within the toroidal structure are an X-ray source (not shown) and an array of X-ray detectors (not shown). The X-ray source is aimed substantially at the longitudinal axis and is movable around the interior of the toroidal structure in a plane that is substantially perpendicular to the longitudinal axis. The X-ray detectors are mounted all around the toroidal structure in substantially the same plane as the X-ray source and are aimed at the longitudinal axis. To obtain a CT X-ray image, a patient is placed on the platform and the platform is inserted into the center of the toroidal structure. The X-ray source then rotates around the patient continuously emitting X-rays and the detectors sense the X-ray radiation that passes through the patient. Since the detectors are in the same plane as the X-ray source, the signals they receive relate essentially to a slice through the patient's body where the plane of the X-ray source and detectors intersect the body. The signals from the X-ray detectors are then processed by the computer to generate an image of this slice known in the art as an axial section.

As an example, X-rays may be emitted continuously for the full 360° around the patient and numerous features are observed but the overall approach is generally the same.

While the patient remains motionless, the platform is moved along the longitudinal axis through the toroidal structure. In the course of this movement, X-ray exposures are continuously made of the portion of the patient on which CT is to be performed. Since the table is moving during this process, the different X-ray exposures are exposures of different slices of the portion of the patient being examined and the images generated by the computer are a series of axial sections depicting in three dimensions the portion of the patient's body that is being examined. The spacing between adjacent CT sections depends on the minimum size of the features to be detected. For detection at the highest resolution, center-to-center spacing between adjacent sections should be on the order of less than 2 mm.

Because of the superior imaging capabilities of CT, the use of CT in medical imaging has grown rapidly in the last several years due to the emergence of multi-slice CT. One application of medical CT is detection and confirmation of cancer. The diagnostically superior information now available in CT axial sections, especially that provided by multidetector CT (multiple slices acquired per single rotation of the gantry) where acquisition speed and volumetric resolution provide exquisite diagnostic value, however, enables the detection of potential cancers at the earliest and most treatable stage. For example, the minimum detectable size of a potentially cancerous nodule in an axial section of the lung is about 2 mm ( 1/10 of inch), a size that is potentially treatable and curable if detected.

Recently, medical professionals have been able to diagnose lung cancer with the aid of computed tomography (CT) imaging systems. Radiologists are able to examine these series of cross sectional images to diagnose pulmonary nodules. The radiologists' examinations also diagnose whether these pulmonary nodules are malignant or benign. If a radiologist confirms confidently that a pulmonary nodule is benign, further medical examination may be avoided.

To enable accurate diagnosis of pulmonary nodules that have the size around the resolution of the CT scanner, it may be advantageous to combine the CT scan with a computer-aided diagnostic (CAD) scheme to assist radiologists.

A procedure in accordance with the present disclosure may be performed with CT guidance. CT is particularly well suited for solid organ interventions. With CT fluoroscopy, which shows the motion of organs and devices in real time, the trajectory of a needle may be tracked in real time, which allows the physician to make adjustments as appropriate. This advantage has made procedures shorter with equivalent or better success rates than those with standard intermittent CT imaging.

A CT scan be used to locate target sites for the anchor. CT scans may be used to reconstruct the 3D positioning of the target site with respect to fiducial markers on the body of the patient. This reconstructed 3D image of CT slices may be loaded to a system that helps the physician navigate the devices of the present disclosure through the patient's body and/or help determine the best route for access.

The devices of the present disclosure may be fitted with an accelerometer and/or gyroscope that helps determine the position of the instrument tip in 3D space at all times. By enabling communication between such devices of the present disclosure (fitted with 3D tracking) and the CT software, the tip of the devices of the present disclosure may be determined with respect to the desired target spot. The software may help keep the device on the planned trajectory and help achieve optimal outcomes.

Additionally or alternatively, CT scans may be combined with other imaging modalities, such as ultrasound or electromagnetic tracking of the tip, to facilitate navigation of the devices of the present disclosure.

In an aspect of the present disclosure, a patient may be placed in a CT scanner and the nodule may be imaged. Using standard CT guided interventional techniques commonly used in CT guided biopsy of the lung, an anchor needle may be advanced through the skin, chest wall, pleural space and lung and through to the target tissue to be sampled. Once the distal end of the anchor needle has passed through the nodule or interstitial abnormality, anchoring members comprised of shape memory metal such as Nitinol, are advanced out of the distal end of the needle.

Ultrasound

An ultrasound probe may be used to facilitate detection and/or location of target tissue sites. An ultrasound probe consists of a piezoelectric transducer that generates ultrasonic waves. These ultrasonic waves are reflected differently from various tissues based on their mechanical and constitutional properties. The reflected waves are then acquired through the receiver and interpreted to translate the properties and location of the tissue. By tracking the location of the ultrasound in 3D space, it is possible to generate a 3D map of the tissue imaged using ultrasound.

Alternatively or additionally to providing the location of the specific target tissue sites, ultrasound is also capable of distinguishing tissue stiffness. This is of critical importance as tumors are known for different mechanical and elastic properties than their surrounding tissue. Hence, ultrasound may enable rapid detection and imaging of the tumor site, in addition to providing details on its location, size and other physical properties.

The ultrasound may also be in a probe format that may be inserted into the pleural space, or navigated through the bronchial space. The probe may be in the form of a catheter configured to facilitate visualization. Such a catheter may be rotated continuously to get a complete 360 ultrasound map as the catheter navigates through the space (iVUS).

The tip has a lubricious covering that allows the operator to run the ultrasound probe over the surface of the lung until the nodule is localized. Once the nodule is localized, a suction apparatus around the perimeter of the ultrasound probe may be actuated so that the lung is sucked into the scope/probe, thus securing the area and locking the probe into place. A needle may be advanced through the lung (e.g., by an operatory) under ultrasound guidance to access the nodule.

MRI/Magnetic Detection

MRI or magnetic resonance imaging relies on the use of high flux electromagnets to oscillate polar molecules and thereby image the localization of those polar molecules. The most ubiquitous polar molecule is water present in human tissue. The water content of normal tissue is different from tumor tissues. For example, tumors usually have elaborate blood supply and drainage, compared to normal tissue. This may be used to visualize a target tissue site. Depending on the target tissue properties, a contrast agent may be added to enhance the resolution of the imaging technique. The contrast agent may comprise components that have a high dipole moment or respond, through motion, emission or vibration, to changes in surrounding magnetic fields.

Endoscope

An endoscope may be used to facilitate visualization of a target tissue site. Specifically for the lung, endoscopy may be used within the chest, thereby precluding the need for a large thoracotomy incision. Thoracoscopy is the use of a specialized viewing instrument, usually a rigid endoscope, introduced through a thoracostomy, or a small hole placed in between the ribs. Once the endoscope is placed in the space that surrounds the lung, known as the pleural space, additional thoracostomy holes may be made to introduce additional instruments. Additional instruments include grasping instruments, cutting instruments, and/or a cutting stapler, such as the Ethicon Endosurgery Endo GIA 45 mm stapler. Using the endoscope and the other instruments, a “triangulation” technique is utilized where, for example, the endoscope is used to view as the grasping instrument is brought in from one direction, and the stapler is brought in from another, and tissue is cut with the stapler and removed through one of the ports.

Visual

Visual imaging may be done using the following modalities: Laser Doppler perfusion imaging (LDPI), Laser speckle contrast imaging (LSCI), Tissue viability imaging (TiVi), Photoacoustic Imaging (PAI), Optical coherence tomography (OCT), Infrared based imaging, optical camera

A wide range of visualization techniques may be used for detection and imaging of the target tissue site. These techniques employ a certain wavelength range or combination of multiple wavelengths to yield deterministic results. Depending on the wavelength range used by the source, the penetration depth may vary and therefore, it is possible to image the target tissue site non-invasively. The light (radiation source) could be a hand held probe that is used scan the patient's body from exterior, similar to an ultrasound probe, for visualization or detection of the target tissue site. Alternatively, the light source could be mounted on a probe and navigated through the patient's body up to a point close enough to visualize the target tissue site. Such a probe could be advanced through the pleural cavity along the trachea and used to detect or visualize the target tissue in the lungs.

These imaging techniques could be combined with other imaging modalities, such as ultrasound, electrical detection, etc., to enhance the resolution.

Additionally, external agents may be administered, such as contrast, nanoparticles, fluorescing agents, etc., to enhance the resolution or detection capabilities of visual imaging techniques.

Electromagnetic/Electrical Potential/Impedance

An electromagnetic probe may be used to visualize the target tissue site.

An electromagnetic guided probe may also be used to remotely control the navigation to the target tissue site.

A probe capable of detecting differences in zeta potential changes as it is navigated through the tissue may be used for detection and visualization of the target tissue site.

Bioimpedance analysis relates to the measurement that an organ or tissue responds additional applied current. The bio-impedance parameter that may record is as resistance, reactance, phase angle, and it is to determine for the purpose of blood flow and body composition (such as, water and fat content). However, there is the physical evidence of accumulation, at least the phase angular dimensions of bioimpedance analysis measures at body composition, as general health situation index and forecasting tool likely exceeded its stage generally used. Phase angle it has been generally acknowledged that, such as, be cell membrane integrity and the fluid index in the intra or extracellular spatial distribution of cellular level. Ongoing research shows, phase angle also may reflect other biological attribute.

Based on Cole-Cole model and Hanai method, a kind of method of bio-impedance frequency spectrum (BIS) of utilizing has been proposed to be used in measurement extracellular liquid volume (ECV) and intracellular fluid body volume (ICV). Now, multi-frequency bioimpedance analysis method may provide some information about extracellular fluid and intracellular fluid volume in health compartment total or sections.

The ability of recognizing cancer cells using bioimpedance is well established in the biomedical literature. The usual method for measuring bioimpedance is by introducing a sample into a special chamber and applying an AC current through it while recording the voltage across the sample at each frequency. More modern methods rely on multiple electrode matrices which are connected with the human body and measure physiological and pathological changes. Some of the methods aim to localize tumor cells inside the human body and to form an image. Another technique, based on magnetic¹³ bioimpedance, measures the bioimpedance by magnetic induction. This technique consists of a single coil acting as both an electromagnetic source and a receiver operating typically in the frequency range 1-10 MHz. When the coil is placed in a fixed-geometric relationship to a conducting body, the alternating electric field in the coil generates electrical eddy current. A change in the bioimpedance induces changes in the eddy current, and as a result, a change in the magnetic field of those eddy currents. The coil acts as a receiver to detect such changes. Experiments with this technique achieved sensitivity of 95%, and specificity of 69%, distinguishing between 1% metastasis tumor and 20% metastasis tumor. Distinguishing between tumor and normal tissue is even better.

X-ray

X-rays are electromagnetic radiation with high penetration capabilities.

Differences in elemental properties of tissues will pose differences in resistance to X-ray radiation. This property of the target tissue may be used to detect and visualize the target tissue site.

Fiducial markers, comprised of material opaque to X-rays, for example, lead, may be placed on the patient's body to aid navigation to the target tissue and for trajectory planning.

Navigation Systems

Various systems, devices, and apparatus may be used to navigate instruments and/or devices to a target site such as a target tissue site in a human body. For example, navigation systems may be used such as Auris, robotic, CT/ultrasound fusion, electromagnetic navigation, fluoroscopic, etc.

Auris

Auris is a system and tools for endolumenal robotic procedures that provide improved ergonomics, usability, and navigation. Endoscopy is a widely-used, minimally invasive technique for both imaging and delivering therapeutics to anatomical locations within the human body. Typically a flexible endoscope is used to deliver tools to an operative site inside the body—e.g., through small incisions or a natural orifice in the body (nasal, anal, vaginal, urinary, throat, etc.)—where a procedure is performed. Endoscopes may have imaging, lighting and steering capabilities at the distal end of a flexible shaft enabling navigation of non-linear lumens or pathways.

Auris typically uses a sheath with a lumen, having a controllable and articulable distal end, which is mounted to a first robotic arm having at least 3 DOF, but preferably 6 or more DOF. This embodiment also includes a flexible endoscope having a controllable and articulable distal end, a light source and video capture unit at the distal end thereof, and at least one working channel extending. The flexible endoscope is slidingly disposed in the lumen of the sheath, and is mounted to a second robotic arm having at least 3 DOF, but preferably 6 or more DOF. Further included are first and second modules, operatively coupled, respectfully, to the proximal ends of the sheath and flexible endoscope. The modules are mounted to the first and second robotic arms, thereby mounting the sheath and flexible endoscope to first and second robotic arms, respectively. The modules provide the mechanics to steer and operate the sheath and flexible endoscope, and receive power and other utilities from the robotic arms. The robotic arms are positioned such that the first module is distal to the second module and the proximal end of the sheath is distal to the proximal end of the flexible endoscope. Movement of the first and second robotic arms relative to each other and relative to the patient causes movement of the sheath relative to the flexible endoscope and movement of either relative to the patient.

Robotic/Electromagnetic Navigation

Robotically-enabled medical systems may be used to perform a variety of medical procedures, including both minimally invasive procedures, such as laparoscopic procedures, percutaneous and non-invasive procedures, such as endoscopic procedures.

Among endoscopic procedures, robotically-enabled medical systems may be used to perform bronchoscopy, ureteroscopy, gastroenterology, etc. During such procedures, a physician and/or computer system may navigate a medical instrument through a luminal network of a patient. The luminal network may include a plurality of branched lumens (such as in bronchial or renal networks), or a single lumen (such as a gastrointestinal tract). The robotically-enabled medical systems may include navigation systems for guiding (or assisting with the guidance of) the medical instrument through the luminal network. This navigation may be guided using mechanical means, such as that of Auris, or use of electromagnets.

Among percutaneous procedures, robotically-enabled medical systems may be used to perform minimally invasive surgeries. The methods include advancing a first alignment sensor into the cavity through a patient lumen. The first alignment sensor provides its position and orientation in free space in real time. The alignment sensor is manipulated until it is located in proximity to the object. A percutaneous opening is made in the patient with a surgical tool, where the surgical tool includes a second alignment sensor that provides the position and orientation of the surgical tool in free space in real time. The surgical tool is directed towards the object using data provided by both the first and the second alignment sensors.

The alignment sensor may, for example, be an anchor coupled with an EM sensor which works in conjunction with EM field generators placed around the patient and an associated CT (or other) scan to provide position and orientation information for EM sensor in the patient's body. The alignment sensor is placed via a cavity, such as the devices of the present disclosure, and together with a camera is used to identify the location of the target tissue site. The alignment sensor provides a guidance mechanism for directing the percutaneous cut for accessing the target tissue site within lungs. Further, as at this point in the procedure, a scope is already present, a working channel of the scope may be used to advance other tools to assist in the removal of the target tissue through a port created by the access devices of the present disclosure.

CT/Fluoroscopy and/or Combining with Ultrasound

Systems and methods are described for navigating a probe to a location within a body of a patient. The probe may comprise a needle, introducer, catheter, stylet, or sheath. Other probes may be used. Methods may comprise visualizing a three-dimensional image of a region of a body of a patient. As an example, the three-dimensional image of a region of a body of a patient may be based on one or more of magnetic resonance imaging (MM), computer tomography (CT), or ultrasound. Other imaging techniques may be used. Methods may comprise receiving a selection of a target location within said three-dimensional image of a region of a patient's body. As an example, the receiving a selection of a target location may be via interaction with a display device configured to output one or more of the visualizing steps. Other inputs may be used to effect selection. Methods may comprise determining and visualizing a preferred pathway for the probe to follow from an external entry point on the patient's body to the target location. The preferred pathway may be determined by transforming a selected point in a two-dimensional view of the three-dimensional image of a region of a body of a patient into a line (e.g., line of sight) through the three-dimensional image of a region of a body of a patient. Methods may further comprise calibrating the preferred pathway to compensate for shift of anatomical structures pre-operatively. Alternatively or additionally, methods may further comprise calibrating the preferred pathway to compensate for shift of anatomical structures intra-operatively. Methods may comprise registering the three-dimensional image to the current actual position of the corresponding region of the patient's body. Methods may comprise registering the current actual position of the probe to the three-dimensional image and the current actual position of the patient's body. Methods may further comprise updating the registration of the three-dimensional image to the patient to compensate for shift of anatomical structures. Methods may comprise visualizing the preferred pathway for the probe simultaneously with an indication of the current actual position of the probe in real time such that the simultaneous visualizations enables a user to align the current actual position of the probe with the preferred pathway. As an example, the indication of the current actual position of the probe may comprise the position of the probe in three-dimensional space. As a further example, the indication of the current actual position of the probe may comprise the projected extension of the probe in three-dimensional space. Methods may comprise updating and visualizing an indication of the current actual position of the probe in real time as the probe is advanced to the target location. Additionally, output of an auditory or visual feedback may be used to warn the user about information regarding proximity to the target location and/or to warn the user about information regarding proximity to critical anatomical structures.

The procedures of the present disclosure may be performed with CT guidance. CT is particularly well suited for solid organ interventions. With CT fluoroscopy, which shows the motion of organs and devices in real time, the trajectory of a needle CT is particularly well suited for solid organ interventions. With CT fluoroscopy, which shows the motion of organs and devices in real time, the trajectory of a needle may be tracked in real-time, which allows the physician to make adjustments as appropriate. This advantage has made procedures shorter with equivalent or better success rates than those with standard intermittent CT imaging.

This advantage has made procedures shorter with equivalent or better success rates than those with standard intermittent CT imaging.

A CT scan be used to locate target sites for the anchor. CT scans may be used to reconstruct the 3D positioning of the target site with respect to fiducial markers on the body of the patient. This reconstructed 3D image of CT slices may be loaded to a system that helps the physician navigate devices of the present disclosure through the patient's body and/or help determine the best route for access.

The devices of the present disclosure may be fitted with an accelerometer and/or gyroscope that helps determine the position of the instrument tip in 3D space at all times. By enabling communication between such as devices of the present disclosure (fitted with 3D tracking) and the CT software, the tip of the devices of the present disclosure may be determined with respect to the desired target spot. The software may help keep the device on the planned trajectory and help achieve optimal outcomes.

Additionally, CT scans may be combined with other imaging modalities, such as ultrasound or electromagnetic tracking of the tip, to facilitate navigation of the devices of the present disclosure.

In an embodiment of the present invention, a patient may be placed in a CT scanner and the nodule may be imaged. Using standard CT guided interventional techniques commonly used in CT guided biopsy of the lung, an anchor needle may be advanced through the skin, chest wall, pleural space and lung and through to the target tissue to be sampled. Once the distal end of the anchor needle has passed through the nodule or interstitial abnormality, anchoring members comprised of shape memory metal such as Nitinol, may be advanced out of the distal end of the needle.

Fluoroscopic

Fluoroscopy uses lower doses of radiation, similar to a CT scanner, to minimize negative effects to the patient.

Anchoring

Various anchor devices may be used. A needle may be anchored to guide the coring device. Non-invasive anchoring may be used. For example, a needle may be advanced to the desired target site via the use of a real time or virtual image guided procedure. The advancing process may be carried out by a person's hands directly, by a person manually using a robotic arm, or autonomously robotically guided per a digital 2D or 3D image. Once the desired position has been achieved, Nitinol fingers may be engaged into the target tissue. Once the Deployment Handle has reached desired location, the Deployment Handle can be removed from the deployed Anchor.

Tissue Site Access

Various systems, devices, and apparatus may be used to provide or support access to a target site such as a target tissue site in a human body. For example, chest wall incision blades, deployable access ports, tissue dilation, trocar, and/or open incisions may be used.

Chest Wall Incision Blades

Once the anchor is placed and deployed at the target location, to access the chest cavity through the chest wall without causing puncture to the lung, there is a need to break the vacuum of the intrapleural space. The chest wall incision blade may be designed with an open channel next to the center hole, which allows the blade to be advanced and cut through chest wall tissue along the anchor. The open channel may be used to allow air to be introduced into the pleural space when the first layer of the pleural space is penetrated. The intrapleural vacuum may be lost, and thus the lung may be dropped away to minimize the potential of damaging to the lung pleura.

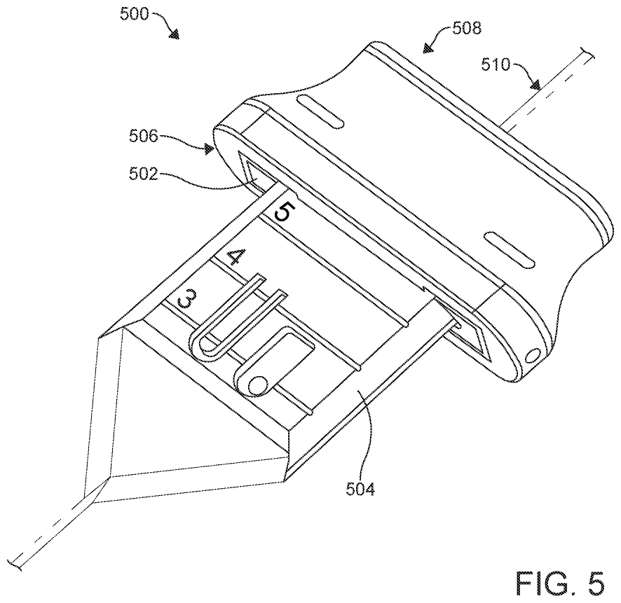

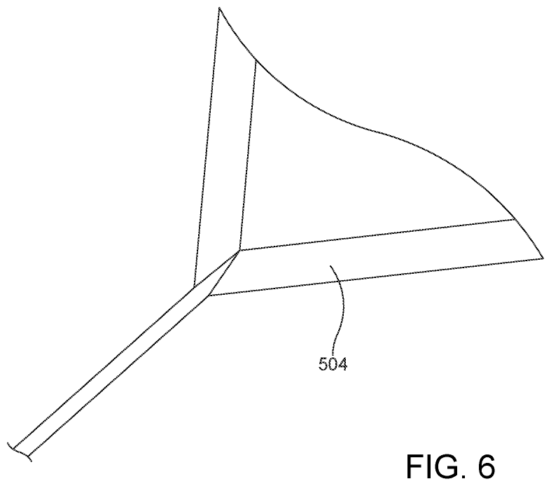

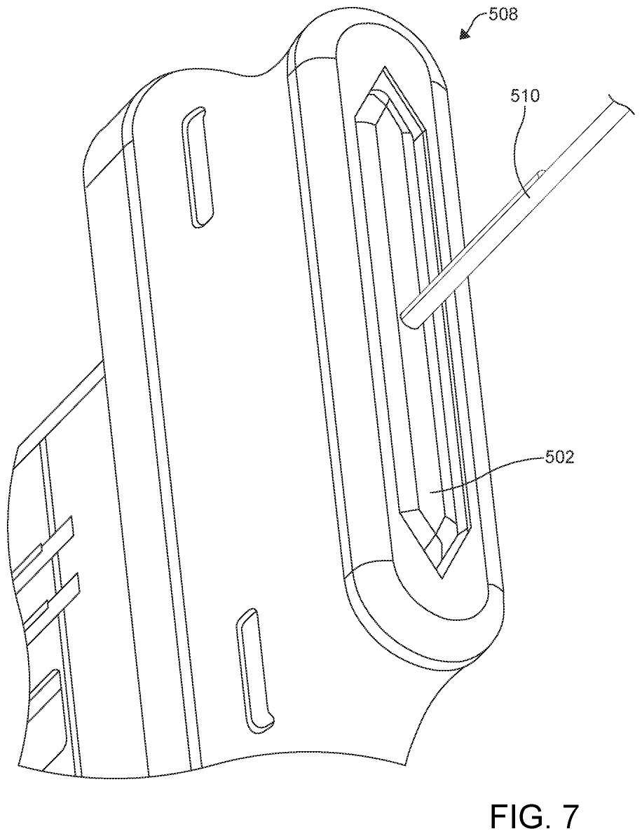

FIG. 5 illustrates a blade device 500 with an open channel 502. The open channel 502 may be an air channel and may be connected to the sharp distal tip 504 of the blade at a distal end 506 to allow air to continuously flow to the distal tip 504 of the blade device 500 (see, e.g., FIG. 6). FIG. 6 illustrates the distal tip 504 of the blade of FIG. 5. The proximal end 508 of the open channel 502 may be connected to a rigid or flexible tube 510. Air may enter the open channel 502 by ambient pressure or by a higher pressurized air (see, e.g., FIG. 7). FIG. 7 illustrates the proximal end 508 of the open channel 502 connected to the flexible or rigid tube 510.

Cavity Access Sleeve

Post coring and amputation of the target tissue, prior to removing the coring device with the target tissue inside, a cavity access sleeve may be placed on the outside diameter of the coring device shaft to maintain access to the location where the target tissue was removed from. Re-access to the location may be desirable for post coring treatment, such as adding a marking device of the tissue location for subsequent surgery, cavity seal, cavity ablation, delivery of drug or local chemotherapy. Without placing a cavity access sleeve prior to removing the coring device, re-access to the removed target tissue location could be difficult in an organ that has large movement, such as the lung.

Tissue Dilation

After the anchor is deployed at a target tissue location of an organ, such as a target lesion in a human lung, to spare the healthy tissue between the organ surface and the target tissue from being removed, the tissue may be dilated to allow subsequent insertion of the coring device to remove the target tissue only. The dilation may be achieved as follows:

Rigid rods with center holes may be advanced over the anchor until the distal ends of the rods reach the target tissue. The rigid rods may have a diameter increasing from small to larger diameters.

An expandable rod may be advanced over the anchor until the distal end of the expendable rod reaches the target tissue. At this point, the distal end of the rod may be expanded to a desired diameter.

A balloon catheter in its collapsed state may be advanced over the anchor. Once the distal end of the balloon catheter reaches the target site, the balloon may be expanded to dilate the tissue. The balloon may have a similar shape as an angioplasty balloon, or it may be configured to have square corners at the distal end. Also, the body of the balloon may have features, such as a corrugated balloon, to minimize tissue slippage along the balloon as the balloon is inflated.

Trocar

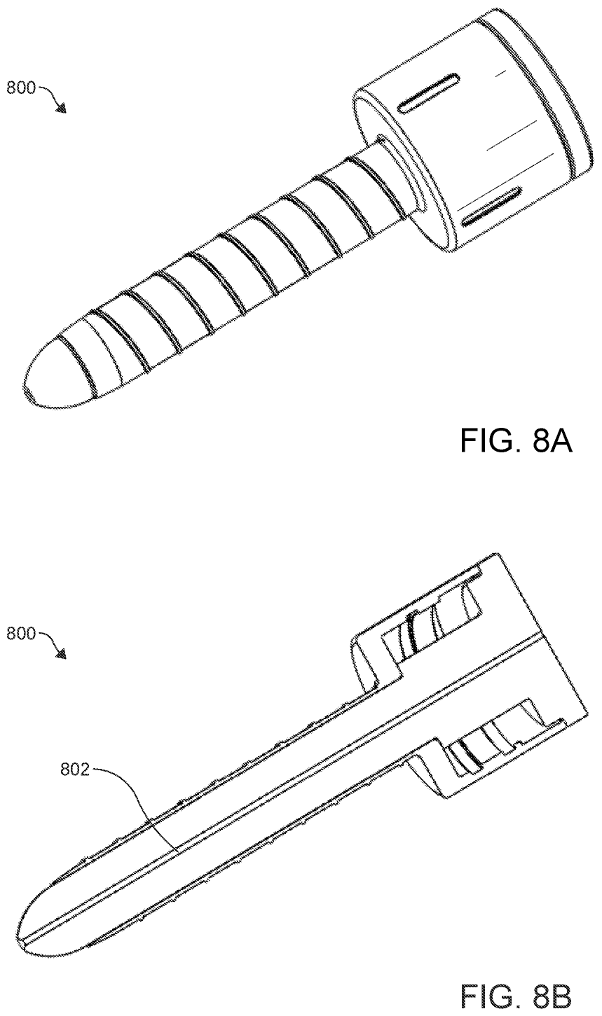

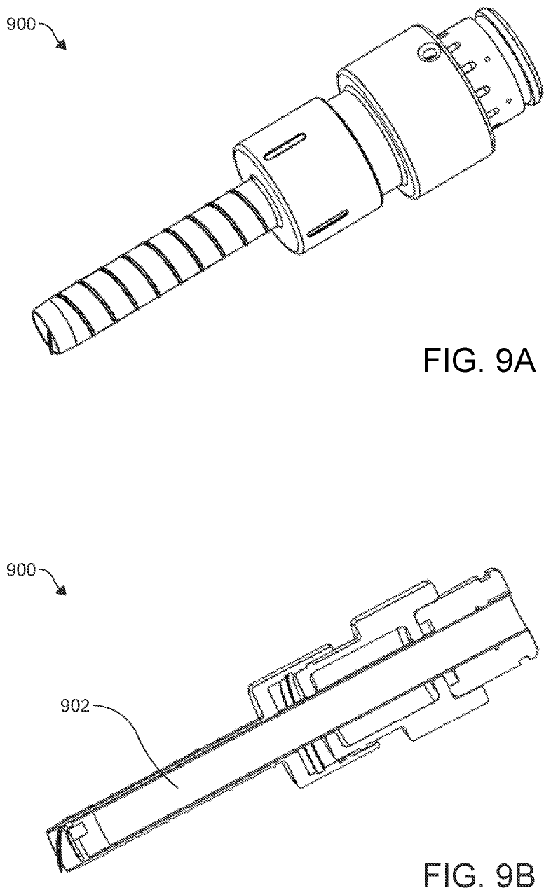

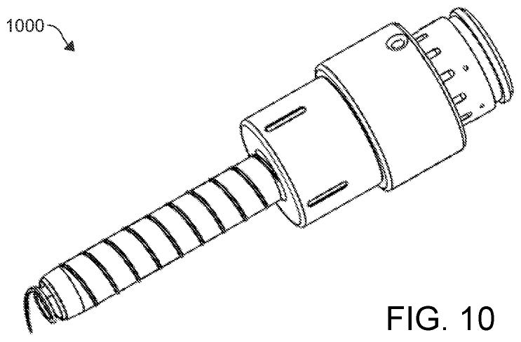

Access to a target tissue site may be achieved via a trocar. Example trocars 800, 900, 1000 are shown in FIGS. 8-10. Trocars may comprise a trocar channel (e.g., trocar channel 802 of FIG. 8B and/or trocar channel 902 of FIG. 9B). Trocar channel may be used to allow air to be introduced into the pleural space when the first layer of the pleural space is penetrated. The intrapleural vacuum may be lost, and thus the lung may be dropped away to minimize the potential of damaging to the lung pleura. Once a lesion has been successfully located, an anchoring device may be used to stabilize the target tissue lesion. The tissue coring device may also be introduced directly to the location of the target lesion using a trocar or under direct visualization with or without a guide anchor and perform the tissue resection.

Open Incision

Access to a target tissue site may be achieved via an open incision. Specifically for the lung, a thoracotomy may be performed and consists of creating a 300 to 450 mm (12 to 18 inches) incision on the chest wall followed by division or dissection of the major back muscles to move them out of the way, partial removal of the rib, and the placement of a rib spreader to provide intra thoracic access to the operating surgeon. The advantage of a thoracotomy is that the surgeon has excellent access to the intrathoracic structures, and may see and manually feel the lung and other structures directly. Once a lesion has been successfully located, an anchoring device (such as the above) may be used to stabilize the target tissue lesion. The tissue coring device may also be introduced directly to the location of the target lesion using an endoscope or under direct visualization with or without a guide anchor and perform the tissue resection.

Tissue Coring

Various methods, devices, and systems may be used to core or remove tissue.

A method for removing a tissue lesion may comprise introducing a tissue resection device to a target tissue site, causing the tissue resection device to resect a core of tissue from the target tissue site, and removing the core of tissue from the body. The core of tissue may comprise at least a portion of a tissue lesion. A method may further comprise creating a core cavity at the target tissue site. A method may further comprise inserting a sleeve into the core cavity. A method may further comprise delivering radiofrequency energy through the core cavity. A method may further comprise delivering chemotherapy through the core cavity. A method may further comprise delivering microwave radiation through the core cavity. A method may further comprise delivering thermal energy through the core cavity. A method may further comprise delivering ultrasonic energy through the core cavity. The tissue resection device may be configured for the delivery of radiofrequency energy. The tissue resection device may be configured for mechanical transection. The tissue resection device may comprise mechanical compression and the delivery of radiofrequency energy. A method may further comprise amputating the core of tissue from the target tissue site. As an example, the means for amputation of the core of tissue may comprise mechanical transection. As a further example, the means for amputation of the core of tissue may comprise the delivery of radiofrequency energy. The means for amputation of the core of tissue may comprise mechanical compression and the delivery of radiofrequency energy. The means for amputation of the core of tissue may comprise transection with an energized wire. Other devices may be used.

A method for removing a core of tissue may comprise introducing a tissue resection device to a target tissue site, causing the tissue resection device to resect a core of tissue from the target tissue site, and removing the core of tissue from the body. A method may further comprise creating a core cavity at the target tissue site. A method may further comprise inserting a sleeve into the core cavity. A method may further comprise delivering radiofrequency energy through the core cavity. A method may further comprise delivering chemotherapy through the core cavity. A method may further comprise delivering microwave radiation through the core cavity. A method may further comprise delivering thermal energy through the core cavity. A method may further comprise delivering ultrasonic energy through the core cavity. The tissue resection device may be configured for the delivery of radiofrequency energy. The tissue resection device may be configured for mechanical transection. The tissue resection device may be configured for mechanical compression and the delivery of radiofrequency energy. A method may further comprise amputating the core of tissue from the target tissue site. The means for amputation of the core of tissue may comprise mechanical transection. The means for amputation of the core of tissue may comprise the delivery of radiofrequency energy. The means for amputation of the core of tissue may comprise mechanical compression and the delivery of radiofrequency energy. The means for amputation of the core of tissue may comprise transection with an energized wire.

A method for removing a core of tissue may comprise introducing a tissue resection device to a target tissue site. The tissue resection device may comprise one or more of: a first clamping element comprising a helical coil and a first electrode, or a second clamping element comprising a second electrode. Where a second clamping element is included, the second clamping element may be positioned to oppose at least a portion of the first clamping element. The method may further comprise causing the tissue resection device to resect a core of tissue from the target tissue site and removing the core of tissue from the body. A method may further comprise creating a core cavity at the target tissue site. A method may further comprise inserting a sleeve into the core cavity. A method may further comprise delivering radiofrequency energy through the core cavity. A method may further comprise delivering chemotherapy through the core cavity. A method may further comprise delivering microwave radiation through the core cavity. A method may further comprise delivering thermal energy through the core cavity. A method may further comprise delivering ultrasonic energy through the core cavity. The tissue resection device may be configured for resecting the core of tissue comprises the delivery of radiofrequency energy. The tissue resection device may be configured for resecting the core of tissue comprises mechanical transection. The tissue resection device may be configured for resecting the core of tissue comprises mechanical compression and the delivery of radiofrequency energy. A method may further comprise amputating the core of tissue from the target tissue site. The means for amputation of the core of tissue may comprise mechanical transection. The means for amputation of the core of tissue may comprise the delivery of radiofrequency energy. The means for amputation of the core of tissue may comprise mechanical compression and the delivery of radiofrequency energy. The means for amputation of the core of tissue may comprise transection with an energized wire.

A method for sealing biological fluid vessels may comprise piercing a target tissue site containing a least a portion of at least one target biological fluid vessel with a helical tissue sealing mechanism. The helical tissue sealing mechanism may comprise a helical piercing element and a clamping element. The method may comprise causing the helical tissue sealing mechanism to apply mechanical compression to at least one target biological fluid vessel and delivering energy to seal at least one target biological fluid vessel. The helical piercing element may comprise the clamping element. The mechanical compression may be applied between the helical piercing element and the clamping element. A method may further comprise a second clamping element. The mechanical compression may be applied between the first and second clamping elements. The delivered energy may comprise monopolar radiofrequency energy. The delivered energy may comprise bipolar radiofrequency energy. The delivered energy may comprise thermal energy. The delivered energy may comprise ultrasonic energy.

A method for sealing biological fluid vessels may comprise piercing a target tissue site with a helical piercing element, adjusting the pitch of the helical piercing element to apply mechanical compression to the target tissue, and delivering energy to seal at least one biological fluid vessel in the target tissue. The helical piercing element may comprise a plurality of tissue sealing electrodes. The delivered energy may comprise monopolar radiofrequency energy. The delivered energy may comprise bipolar radiofrequency energy. The delivered energy may comprise thermal energy. The delivered energy may comprise ultrasonic energy.

A tissue resection apparatus may comprise a first clamping element comprising a helical coil, a second clamping element, the second clamping element being positioned to oppose at least a portion of the first clamping element, a first and second electrode configured for the delivery of radiofrequency energy for sealing tissue, and a cutting element configured for the transection of at least a portion of the sealed tissue. A tissue resection device may further comprise: a first actuator operable to actuate the first or second clamping element to apply mechanical compression to tissue and a second actuator operable to actuate the cutting element to transect tissue. The helical coil may include first and second contiguous coil segments. The first coil segment may comprise a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised of at least a portion of the first clamping element. The second electrode may be comprised of at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprises a suture. The tissue resection device may further comprise an actuator operable to actuate the second cutting element to transect tissue.

A tissue resection apparatus may comprise a first clamping element having a helical coil disposed on a distal end, a second clamping element, the second clamping element being positioned to oppose at least a portion of the first clamping element, a first and second electrode configured for the delivery of radiofrequency energy for sealing tissue, and a cutting element configured for the transection of at least a portion of the sealed tissue. The tissue resection device may further comprise a first actuator operable to actuate the first or second clamping element to apply mechanical compression to tissue and a second actuator operable to actuate the cutting element to transect tissue. The helical coil may comprise first and second contiguous coil segments. The first coil segment may comprise a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised of at least a portion of the helical coil. The first electrode may be comprised of at least a portion of the first clamping element. The second electrode may be comprised of at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprise a suture. The tissue resection device may further comprise an actuator operable to actuate the second cutting element to transect tissue.

A tissue resection apparatus may comprise a first clamping element comprising a helical coil and a first electrode, and a second clamping element comprising a second electrode, the second clamping element being positioned to oppose at least a portion of the first clamping element. The first and second clamping elements may be configured for: (a) the delivery of radiofrequency energy for sealing tissue, and (b) the application of mechanical compression for the transection of tissue. The tissue resection device may further comprise a first actuator operable to actuate the first or second clamping element to apply mechanical compression to tissue and a second actuator operable to actuate the cutting element to transect tissue. The helical coil may comprise first and second contiguous coil segments. The first coil segment may comprise a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised by at least a portion of the helical coil. The first electrode may be comprised of at least a portion of the first clamping element. The second electrode may be comprised of at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprise a suture. The tissue resection device may further comprise an actuator operable to actuate the second cutting element to transect tissue.

A surgical instrument system for the resection of tissue may comprise an end effector operable to cut and seal tissue, wherein the end effector and a generator configured to provide power to the end effector having the first and second electrodes for sealing tissue. The end effector may comprise a first clamping element comprising a helical coil, a second clamping element, the second clamping element being positioned to oppose at least a portion of the first clamping element, a first and second electrode configured for the delivery of radiofrequency energy for sealing tissue, and a cutting element configured for the transection of at least a portion of the sealed tissue. The surgical instrument system may further comprise a controller in communication with the generator, wherein the controller is configured to control the generator to provide radiofrequency energy sufficient to seal tissue to the first and second electrodes of the end effector, based on at least one sensed operating condition of the end effector. The controller may be configured to sense the presence of tissue at the end effector. The controller may be configured to sense the presence of tissue at the end effector based on a measured impedance level associated with the first and second electrodes. The controller may be configured to sense an amount of force applied to at least one of the first or second clamping elements to detect the presence of tissue at the end effector. The controller may be configured to sense the position of the cutting element relative to at least one of the first or second clamping elements. The controller may be configured to control the generator to provide radiofrequency energy at the end effector when the second actuator is actuated and no tissue is sensed at the end effector. The controller may be configured to control the generator to provide a continuous amount of radiofrequency energy. The controller may be configured to control the generator to automatically provide an increase or decrease in the amount of radiofrequency energy. The system may further comprise a first actuator operable to actuate the first or second clamping element to apply mechanical compression to tissue, and a second actuator operable to actuate the cutting element to transect tissue. The helical coil may comprise first and second contiguous coil segments, the first coil segment including the first electrode. The first coil segment may comprise a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised of at least a portion of the helical coil. The first electrode may be comprised of at least a portion of the first clamping element. The second electrode may be comprised of at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprise a suture. The tissue resection device may further comprise an actuator operable to actuate the second cutting element to transect tissue.

A tissue resection apparatus may comprise a first clamping element comprising a helical coil, a second clamping element, the second clamping element being positioned to oppose at least a portion of the first clamping element, a first and second electrode configured for the delivery of radiofrequency energy for sealing tissue, a first cutting element configured for the transection of at least a portion of the sealed tissue, a first and second ligating element, and a second cutting element positioned between said first and second ligating elements. The tissue resection device may further comprise a first actuator operable to actuate the first or second clamping element to apply mechanical compression to tissue, and a second actuator operable to actuate the cutting element to transect tissue. The helical coil may comprise first and second contiguous coil segments. The first coil segment may comprise a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised of at least a portion of the helical coil. The first electrode may be comprised of at least a portion of the first clamping element. The second electrode may be comprised of at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprise a suture. The tissue resection device may further comprise an actuator operable to actuate the second cutting element to transect tissue.

A tissue sealing mechanism may comprise a helical coil with a generally obround cross section and a tapered point disposed at a distal end, a first and second helical tissue sealing surface, wherein the first and second helical tissue sealing surfaces are provided by the parallel planar surfaces of the helical coil, a first electrode disposed on the first helical tissue sealing surface, and a second electrode disposed on the second helical tissue sealing surface, wherein the first and second electrodes are configured to apply bipolar radiofrequency energy for sealing tissue. The helical coil may comprise first and second contiguous coil segments. The helical coil may comprise a blunt tip. The first and second electrodes may have surface profiles that are substantially matching. The first and second helical tissue sealing surfaces may further comprise a plurality of electrodes configured for the delivery of bipolar radiofrequency energy.

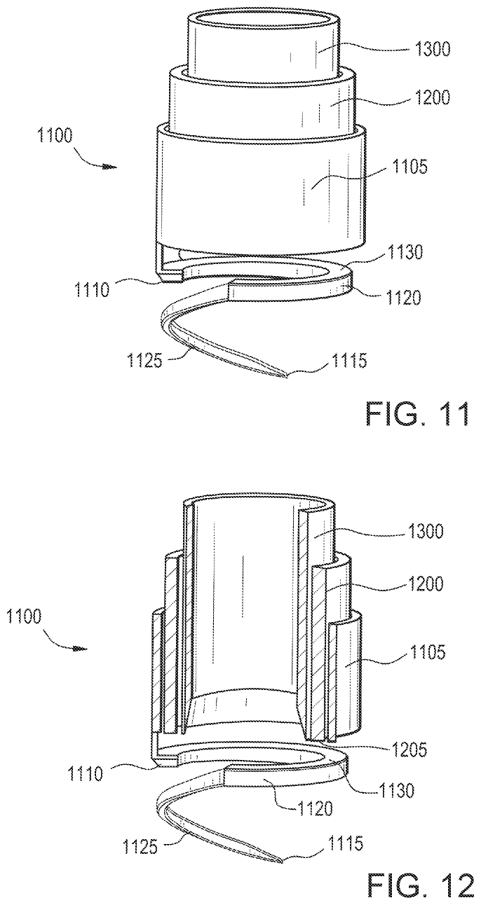

FIGS. 11-17 shown examples devices that may be used to effect a coring process, as described herein. For example, a resection device of the present invention may comprise an energy-based arrangement capable of penetrating tissue towards a target lesion. In one embodiment depicted in FIG. 11, tissue resection device 1100 includes an outer tube 1105 may be provided having a distal edge profile and having an inner diameter IDouter. A coil 1110 may be attached to an outer tube 1105 where the coil turns are spaced from and opposed to a distal end of the outer tube 1105. The coil 1110 preferably has a slightly blunted tip 1115 to minimize the possibility that it will penetrate through a blood vessel while being sufficiently sharp to penetrate tissue such as pleura and parenchyma. In some embodiments, the coil 1110 may take the form of a helix having a constant or variable pitch. The coil 1110 may also have a variable cross-sectional geometry. An electrode 1130 may be disposed on a surface or embedded within the coil 1110.

In some embodiments, as illustrated in FIG. 11, the coil 1110 may include a plurality of contiguous coil segments, e.g., coil segments 1120 and 1125. The coil segment 1120 may comprises a helical member having a pitch of zero, e.g., a generally planar open ring structure, having an inner diameter IDcoil and an outer diameter ODcoil. The coil segment 1125 may comprise a helical structure of constant or variable pitch and constant or variable cross-sectional geometry. In this embodiment, the electrode 1130 may be disposed on a surface of or embedded in the coil segment 1120.

A central tube 1200 may be provided having a distal end with an edge profile comprising one or more surface segments and having an outer diameter ODcentral and an inner diameter IDcentral. As illustrated in FIG. 12, an electrode 1205 may be disposed on or embedded within at least one of the surface segments. The central tube 1200 may be slidably disposed within the outer tube 1105 and positioned such that the electrode 1205 opposes and overlaps at least a portion of electrode 1130. The space between electrode 1205 and electrode 1130 may be referred to as the tissue clamping zone. In keeping with an aspect of the present disclosure, ODcentral>IDcoil and ODcoil>IDcentral. In some embodiments, ODcentral may be about equal to ODcoil. Accordingly, the central tube 1200 may be advanced through the tissue clamping zone towards coil 1110 such that electrode 1205 abuts electrode 1130.

A cutting tube 1300 may be slidably disposed within the central tube 1200. The distal end of the cutting tube 1300 may be provided with a knife edge to facilitate tissue cutting.

To enable tissue resection, the resection device 1100 may be inserted into tissue and the outer tube 1105 may be advanced a predetermined distance towards a target. The coil segment 1125 may allow the device to penetrate the tissue in a manner similar to a cork screw. As the coil segment 1125 penetrates tissue, any vessel in its path may either be moved to planar coil segment 1120 or pushed away from the coil 1100 for subsequent turns. A coil tip 1115 may be made blunt enough to minimize chances that it will penetrate through a blood vessel, while still sharp enough to penetrate certain tissue, such as the lung pleura and parenchyma. The central tube 1200 may then be advanced a predetermined distance towards the target. Any vessels that are disposed in the tissue clamping zone will be clamped between electrode 1130 and electrode 1205. The vessels may then be sealed by the application of bipolar energy to electrode 1130 and electrode 1205. Once blood vessels are sealed, the cutting tube 1300 may be advanced to core the tissue to the depth that the outer tube 1105 has reached. The sealing and cutting process may be repeated to create a core of desired size.

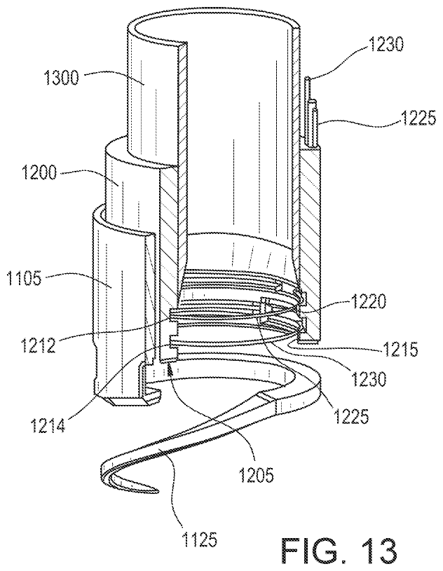

In keeping with an aspect of the present disclosure, the resection device may be further configured to dissect a target lesion and seal tissue proximate the dissection point. To facilitate dissection and sealing, as illustrated in FIG. 13, the central tube 1200 may be provided with a ligation snare 1230, first and second ligation electrodes 1215 and 1220, and an amputation snare 1225. As used herein, the word “snare” refers to a flexible line, e.g., a string or a wire. The inner wall surface of the central tube 1200 may include upper and lower circumferential grooved pathways 1212 and 1214 disposed proximate the distal end. The first and second ligation electrodes 1215 and 1220 may be disposed on the inner wall of central tube 1200 such that lower circumferential groove 1214 may be between them. The upper grooved pathway 1212 may be disposed axially above the ligation electrodes 1215 and 1220.

The ligation snare 1230 may be disposed in the lower circumferential groove 1214 and extends through the central tube 1200 and axially along the outer wall surface to a snare activation mechanism (not shown). The amputation snare 1225 may be disposed in the upper circumferential groove 1212 and extends through the central tube 1200 and axially along the outer wall surface to a snare activation mechanism (not shown). The outer surface of the central tube 1200 may be provided with a plurality of axially extending grooved pathways which receive the amputation snare 1225 and the ligation snare 1230 and are in communication with the upper and lower circumferential grooved pathways 1212 and 1214. In addition, electrode leads for the ligation electrodes 1215 and 1220 may extend to an energy source via the axially extending grooved pathways.

In operation, the resection device of this embodiment may detach and seal the tissue core. The cutting tube 1300 may be retracted to expose the ligation snare 1230 which may be preferably made of flexible line, e.g., suture. The ligation snare 1230 may be engaged to snag tissue and pull tissue against the inner wall surface between the first and second ligation electrodes 1215 and 1220. Bipolar energy may then be applied to the first and second electrodes 1215 and 1220 to seal, i.e., cauterize, the tissue. Once sealed, the cutting tube 1300 may be further retracted to expose the amputation snare 1225 which may then be activated to sever the tissue core upstream from the point where the tissue was sealed (ligation point). In some embodiments, the amputation snare 1225 has a smaller diameter than that of ligation snare 1230. The smaller diameter facilitates tissue slicing. Accordingly, the resection device 1100 according to this embodiment may both create a tissue core and disengage the core from surrounding tissue.

In an alternative embodiment, the resection device of the present disclosure may be provided with a single snare disposed between ligation electrodes which both ligates and cuts tissue. In this embodiment, the single snare may first pull tissue against the inner wall surface of the central tube 1200 between the ligation electrodes 1215 and 1220. Bipolar energy may then applied to the first and second electrodes 1215 and 1220 to seal, i.e., cauterize, the tissue. Once sealed, the snare may further pulled to sever the tissue core.

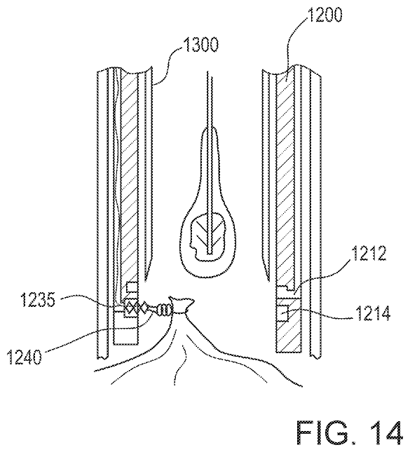

In yet another embodiment, cutting and sealing may be performed without employing electrodes. In this embodiment, the ligation snare 1230 may include a set of knots 1235 and 1240 which tighten under load, shown, for example, in FIG. 14. Ligation may be performed by retracting the cutting tube 1300 to expose the ligation snare 1230 and activating the ligation snare 1230, which lassos tissue as ligation knot tightens. Once the tissue is lassoed, the cutting tube 1300 may be further retracted to expose the amputation snare 1225 which may then be activated to sever the tissue core upstream from the point where the point where the tissue was lassoed.

The present disclosure also contemplates a method and system for using the resection device to remove tissue lesions, for example, lung lesions. The method generally comprises anchoring the lesion targeted for removal, creating a channel in the tissue leading to the target lesion, creating a tissue core which includes the anchored lesion, ligating the tissue core and sealing the surrounding tissue, and removing the tissue core including the target lesion from the channel.

Anchoring may be performed by, any suitable structure for securing the device to the lung. Once the lesion is anchored, a channel may be created to facilitate insertion of the resection device 1100. The channel may be created by making an incision in the lung area and inserting a tissue dilator and port into the incision. A tissue core which includes the anchored lesion may be created. In keeping with the present disclosure, the resection device 1100 may be used to create the tissue core, to ligate the tissue core and to seal the tissue core and sever it from the surrounding tissue as described hereinabove. The tissue core may then be removed from the channel. As an example, a cavity port may be inserted in the channel to facilitate subsequent treatment of the target lesion site through chemotherapy and/or energy-based tumor extirpation such as radiation. As a further example, a cavity port may be disposed on the perimeter of the tissue resection apparatus. When the apparatus is removed from the tissue site, the cavity port may remain in place or may be removed.

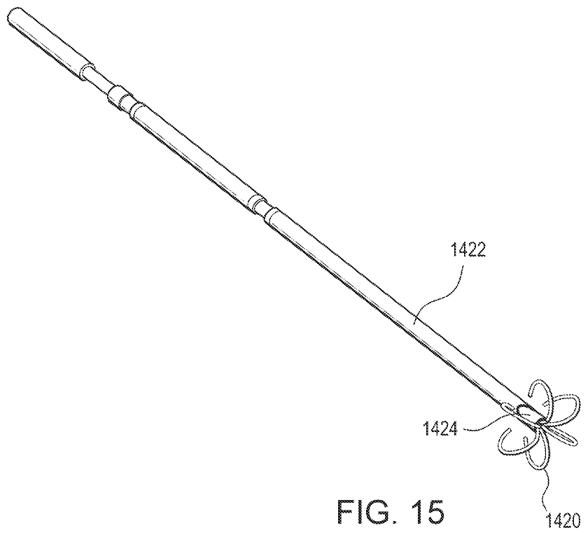

The anchor depicted in FIG. 15 may be suitable for use in performing the method for removing tissue lesions described herein. The anchor may comprise an outer tube 1422 having a sufficiently sharp edge to pierce the chest cavity tissue and lung without causing excess damage and an inner tube 1424 disposed within the outer tube 1422. One or more tines or fingers 1426 formed or preformed from shape memory material, e.g., Nitinol, may be attached to the end of inner tube 1424. The outer tube 1422 may be retractably disposed over the inner tube 1424 such that when the outer tube 1422 may be retracted, the tines 1426 assume their preform shape as shown. In keeping with the present disclosure, the outer tube 1422 may be retracted after it has pierced the lung lesion thereby causing the tines 1426 to engage the lung lesion. Other suitable anchors may include coils and suction-based structures.

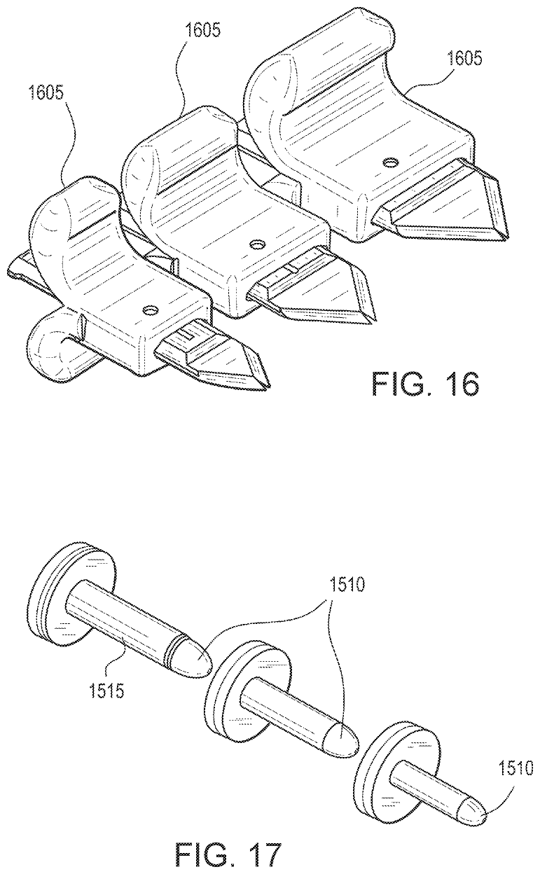

The incision blades depicted in FIG. 16 are suitable for use in performing the method for removing tissue lesions described herein. Once the anchor 1400 is set, it may be preferable to create a small cut or incision to facilitate insertion of chest wall tissue dilator. Incision blades 1605 may be used to make a wider cut. The incision blades 1605 may successive. The incision blades 1605 may include a central aperture which may allow them to be coaxially advanced along the anchor needle 1405 to create a wider cut in the chest wall, with each successive blade being larger than the previous blade, thereby increasing the width of the incision.

The tissue dilator depicted in FIG. 17 may be suitable for use in performing the method for removing tissue lesions described herein. The tissue dilator may comprise any suitable device for creating a channel in organic tissue. In one exemplary embodiment, the tissue dilator assembly includes a single cylindrical rod with a rounded end 1510 or a cylindrical rod with rounded end and a rigid sleeve arrangement 1515. Successive tissue dilators may be coaxially advanced along the anchor needle to create tissue tract or channel in the chest wall, with each successive dilator being larger than the previous dilator, thereby increasing the diameter of the channel. Once a final dilator with rigid sleeve is deployed, the inner rod 1505 may be removed, leaving the rigid sleeve in the intercostal space between ribs to create direct passage to the lung pleura.

Any tissue resection device capable of penetrating lung tissue and creating a tissue core including a target lesion may be suitable for use in performing the method for removing tissue lesions described herein. The tissue resection device 1100 described hereinbefore is preferred.

Once the tissue resection device 1100 is removed, a small channel in the lung may exist where the target lesion was removed. This channel may be utilized to introduce an energy-based ablation device and/or localized chemotherapy depending on the results of the tissue diagnosis. Accordingly, the method and system of the present disclosure may not only be utilized to ensure an effective biopsy is performed but also complete removal of the lesion with minimal healthy lung tissue removal is accomplished.

Generator

Electrical energy applied by the devices of the present disclosure may be transmitted to the devices by a generator. The electrical energy may be in the form of radio frequency (“RF”) energy. In application, an electrosurgical instrument may transmit RF energy through tissue, which causes ionic agitation, or friction, in effect resistive heating, thereby increasing the temperature of the tissue. Because a sharp boundary is created between the affected tissue and the surrounding tissue, surgeons may operate with a high level of precision and control, without sacrificing un-targeted adjacent tissue. The low operating temperatures of RF energy is useful for removing, shrinking, or sculpting soft tissue while simultaneously sealing blood vessels.

The devices of the present disclosure is designed to work with any commercially available bipolar energy generator, such as an Enseal generator or a Bovie generator. The devices of the present disclosure may interface with a “brand-agnostic” generator adapter that enables device operation regardless of the proprietary brand of generator used to delivery radiofrequency energy. In an exemplary embodiment, the adapter may automatically or, with the assistance of a user, manually identify the specific generator product that is connect to any of the devices of the present disclosure. The generator adapter may modify, modulate, or change the output of the generator (which may have subtle characteristic differences depending on the specific generator used) to ensure optimal tissue sealing using the tissue coring devices of the present disclosure. The generator provides radiofrequency power to drive the devices of the present disclosure such as an electrosurgical coring instrument that is used during open or laparoscopic general surgery to cut and seal vessels and to cut, grasp, and dissect tissues. The generator has an Adaptive Tissue Technology, which delivers intelligent energy for greater precision and efficiency.

Sample Analysis

Various systems, devices, processes, and apparatus may be used to analyze a sample such as a cored tissue sample. For example, tissue histology, DNA sequencing, rapid on-site evaluation (ROSE), or a combination of the same may be used. The coring method described provides a large tissue sample. Following the removal of a core of tissue from a site of interest, the specimen may be analyzed for diagnostic purposes using any of the methods described below, independently or in combination.

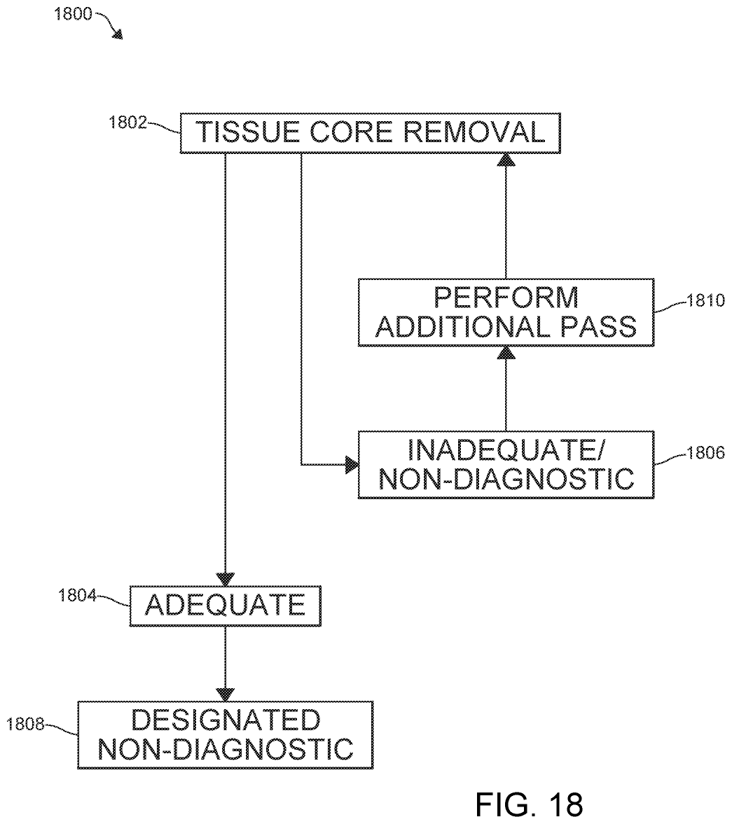

FIG. 18 shows an example workflow 1800 of tissue sample analysis. As illustrated in FIG. 18, tissue sample analysis may further comprise one or more of: removing core tissue (1802) and determining if the removed core tissue is adequate (1804), or inadequate/non-diagnostic (1806). If adequate, the removed tissue core may be analyzed using a designated analysis technique (1808). If inadequate, the workflow may perform an additional pass (1810), and the cycle may continue, starting with step 1802.

Rapid On-Site Evaluation (ROSE)

Rapid on-site examination (ROSE) is a rapid, real-time examination method of the specimen at hand. Use of ROSE during lung lesion biopsy sampling has been suggested to improve diagnostic yield. Reported advantages of ROSE include reduced number of biopsies performed, a lower procedural risk, and an improved accuracy yield. The core of tissue isolated may be analyzed using ROSE techniques. Using ROSE, one may check the sample adequacy and establish a preliminary diagnosis by performing a rapid stain in the bronchoscopy suite or operating room, with evaluation by a cytopathologist or a trained cytotechnologist.

Histology

Morphologic assessment of the core tissue sample may be performed by routine hematoxylin-eosin (H&E) staining, thereby allowing for interpretation of the biopsy.

Immunohistochemistry

A vast majority of neoplasms arising from lung or pleura are initially diagnosed based on the histologic evaluation of tissue biopsies. Although most diagnoses may be determined by morphology alone, immunohistochemistry may be a valuable diagnostic tool in the workup of problematic cases. The core tissue sample may also be analyzed using immunohistochemistry. This may help differentiate between lung adenocarcinoma and squamous cell carcinoma (SqCC), lung adeno-carcinoma and malignant mesothelioma (MM), primary and metastatic carcinomas, and small cell lung carcinoma (SCLC) and carcinoid tumor.

Electron Microscopy

The cored tissue sample may be evaluated using electron microscopy. Electron microscopy may be used to visualize details of a cancer cell's structure that provide clues to the exact type of the cancer.

Flow Cytometry

Flow cytometry is used to detect the presence of tumor markers, such as antigens, on the surface of the cells. It may be used to help in the diagnosis of cancer. The core of tissue isolated may be analyzed using flow cytometry.

Image Cytometry

DNA image cytometry (DNA-ICM) has gained attention for its diagnostic advantages, including objectivity, convenience and a high positive rate, in diagnosing various malignant cancer types. Thus technique has been successfully used for lung biopsies. The core of tissue isolated may be analyzed using image cytometry.

Polymerase Chain Reaction (PCR)

The core of tissue isolated may be analyzed using PCR. PCR may be used to look for certain changes in a gene or chromosome, which may help find and diagnose a genetic condition or a disease, such as cancer.

Gene Expression Microarrays

The core of tissue isolated may be analyzed using gene expression microarrays. Microarray-based technology is an ideal way in which to study the effects and interactions of multiple genes in cancer.

Fluorescent In Situ Hybridization (FISH)

The core of tissue isolated may be analyzed using FISH technology. FISH may be used to identify where a specific gene is located on a chromosome, how many copies of the gene are present, and any chromosomal abnormalities. It is used to help diagnose diseases, such as cancer.

Genetic Sequencing

Next-generation sequencing (NGS) helps to characterize cancer and is rapidly being implemented to guide therapy. It has been previously demonstrated that small lung biopsy samples yield adequate quality DNA and RNA, enabling high-quality NGS analysis. The core of tissue isolated may be analyzed using NGS techniques.

Atomic Force Microscopy

The core of tissue isolated may be analyzed using atomic force microscopy. Atomic force microscopy (AFM) allows for nanometer-scale investigation of cells and molecules. The physicochemical properties of live cells undergo changes when their physiological conditions are altered. These physicochemical properties may therefore reflect complex physiological processes occurring in cells. When cells are in the process of carcinogenesis and stimulated by external stimuli, their morphology, elasticity, and adhesion properties may change. AFM may perform surface imaging and ultrastructural observation of live cells with atomic resolution under near-physiological conditions, collecting force spectroscopy information which allows for the study of the mechanical properties of cells. For this reason, AFM has potential to be used as a tool for the analysis and diagnosis of lung biopsy samples.

Surface Enhanced Ramen Spectroscopy

The core of tissue isolated may be analyzed using surface enhanced Ramen spectroscopy. Ramen spectroscopy may characterize biomolecules, because each macromolecule (lipid, protein, DNA, etc.) has unique finger-printing information about the modes of vibration and rotation. Therefore, Raman spectroscopy may be a promising tool for cancer diagnostics in the future. Nevertheless, Raman spectroscopy has the deficiency of low sensitivity in practical application. Compared with conventional Raman spectroscopy, Raman scattering signals may be strengthened by 4-15 orders of magnitude utilizing surface-enhanced Raman spectroscopy (SERS) technology. Studies have shown that the Raman enhancement effect may be obtained by utilizing silver nanospheres, gold nanospheres, and similar particulates. In clinical detection, label-free SERS detection of tissue provides a rapid and facile way to differentiate tumors from normal tissues. The differences in SERS spectra between lung cancer and normal tissue may be used to potentially diagnose lung cancer.

Sealing

The present disclosure relates to a method to deliver a fill material such as autologous blood to the core site that may be used to seal and provide pneumostasis. As an example, once the tissue specimen is cored and removed from the lung, there may be a need to seal the core site to provide pneumostasis. As a further example, pneumostasis may be achieved in the same surgery session as the tissue removal.

Although autologous blood is described herein as an example, other fill materials and additives may be used. For example, a hemostatic adjunct such as an absorbable gelatin foam (e.g., SURGIFOAM®), biologic, oxidized regenerated cellulose (ORC), fibrin/thrombin spray, etc. As a further example, a patient may have a rare disorder of hemophilia in which their blood does not clot normally. Other patients may be on blood thinning medicines which could inhibit blood clotting formation. For such patients, to seal the cored cavity, thrombin and/or fibrinogen may be added to the autologous blood sample to aid in clot formation. Reactive polyethylene glycol (PEG), ammonium sulfate, ethanol, calcium chloride, or magnesium chloride may also be added to the blood sample to aid in clot formation. Another source for the blood to be used to seal the cored cavity is donated blood from other people or blood bank. Donated blood may be used with or without clotting agents as mentioned above.

Systems and/or methods for sealing tissue are described herein. An example method may comprise disposing a port to provide access to a target site. The target site may comprise biological tissue. The target site may comprise tissue of a lung. The target site may comprise a cored tissue. The target site may comprise a punctured tissue. Other sites may benefit from the disclosed methods.

Example methods may comprise anchoring an anchor device (e.g., via the port) to a surface at the target site. Anchoring may be performed by any suitable structure for securing the device to the lung. Example methods may comprise disposing (e.g., via the port) a sealing device adjacent the target site. Example methods may comprise disposing a sealing device adjacent the target site using the anchoring device as a guide. The sealing device may comprise an inflatable balloon. The sealing device may comprise an inflatable balloon with an array of radio frequency (RF) electrodes configured to ablate and seal tissue. The sealing device may comprise an inflatable balloon configured to seal tissue using a thermal fluid. The sealing device may comprise an inflatable balloon catheter. The sealing device may comprise an access port with an array of RF electrodes configured to ablate and seal tissue. The sealing device may comprise at least one microwave ablation probe.

Example methods may comprise causing the sealing device to seal the target site. The causing the sealing device to seal the target site may comprise causing at least a portion of the sealing device to abut a portion of the target site. Example methods may comprise disposing a fill material adjacent the target site. Example methods may comprise disposing a fill material adjacent the target site via a fill material delivery device such as a catheter. The fill material may comprise autologous blood, donated blood, recirculated blood, hemostatic adjuncts such as fibrin and/or thrombin, biological tissue adhesives such as Dermabond®, ORC, absorbable gelatin, or any combination thereof. The fill material may promote pneumostasis. The fill material may additionally promote hemostasis. Other materials may be used. The sealing device may minimize escape of the fill material from the target site.

As an illustrative example, the target site may comprise at least a portion of a lung. The lung may be caused to collapse prior to disposing the sealing device adjacent the target site. The lung may be allowed to ventilate while the sealing device is sealing the target site. The sealing device may be spaced (e.g., removed, separated, etc.) from the target site after the fill material is disposed.

Systems and/or methods for sealing are described herein. An example method may comprise disposing a sealing device adjacent a target site of a lung. The sealing device may be disposed adjacent the target site while the lung is collapsed. However, the lung may be ventilated. Example methods may comprise causing the sealing device to seal the target site. Example methods may comprise disposing a sealing device adjacent the target site using the anchoring device as a guide. The sealing device may comprise an inflatable balloon. The sealing device may comprise an inflatable balloon with an array of RF electrodes configured to ablate and seal tissue. The sealing device may comprise an inflatable balloon configured to seal tissue using a thermal fluid. The sealing device may comprise an inflatable balloon catheter. The sealing device may comprise an access port with an array of RF electrodes configured to ablate and seal tissue. The sealing device may comprise at least one microwave ablation probe. Example methods may comprise disposing a fill material adjacent the target site. Example methods may comprise disposing a fill material adjacent the target site via a fill material delivery device such as a catheter. The fill material may comprise autologous blood, donated blood, recirculated blood, hemostatic adjuncts such as fibrin, thrombin, biological tissue adhesives such as Dermabond®, ORC, absorbable gelatin, or any combination thereof. The fill material may promote pneumostasis. The fill material may additionally promote hemostasis. Other materials may be used. The sealing device may minimize escape of the fill material from the target site.

Systems and/or methods for sealing are described herein. An example method may comprise disposing a fluid delivery device into a target site of a lung. The sealing device may be disposed adjacent the target site while the lung is collapsed. However, the sealing device may be disposed adjacent the target site when the lung is ventilated. Example methods may comprise disposing a fill material into the target site. Example methods may comprise spacing (e.g., removing, separating, etc.) the sealing device from the target site.

The sealing device may comprise an inflatable balloon. The sealing device may comprise an inflatable balloon with an array of RF electrodes configured to ablate and seal tissue. The sealing device may comprise an inflatable balloon configured to seal tissue using a thermal fluid. The sealing device may comprise an inflatable balloon catheter. The sealing device may comprise an access port with an array of RF electrodes configured to ablate and seal tissue. The sealing device may comprise at least one microwave ablation probe. The systems and/or methods described herein may allow clotted blood to provide a seal to achieve pneumostasis. Example methods may comprise disposing a fill material adjacent the target site. Example methods may comprise disposing a fill material adjacent the target site via a fill material delivery device such as a catheter. The fill material may comprise autologous blood, donated blood, recirculated blood, hemostatic adjuncts such as fibrin, thrombin, biological tissue adhesives such as Dermabond®, ORC, absorbable gelatin, or any combination thereof. The fill material may promote pneumostasis. The fill material may additionally promote hemostasis. Other materials may be used. The sealing device may minimize escape of the fill material from the target site.

The target site may comprise a cavity. The cavity may be closed, for example, after sealing. Closing the cavity may comprise using biological tissue adhesive such as Dermabond®, tissue grafts, hemostatic sealing patches, staple closure, sutures, or the like.

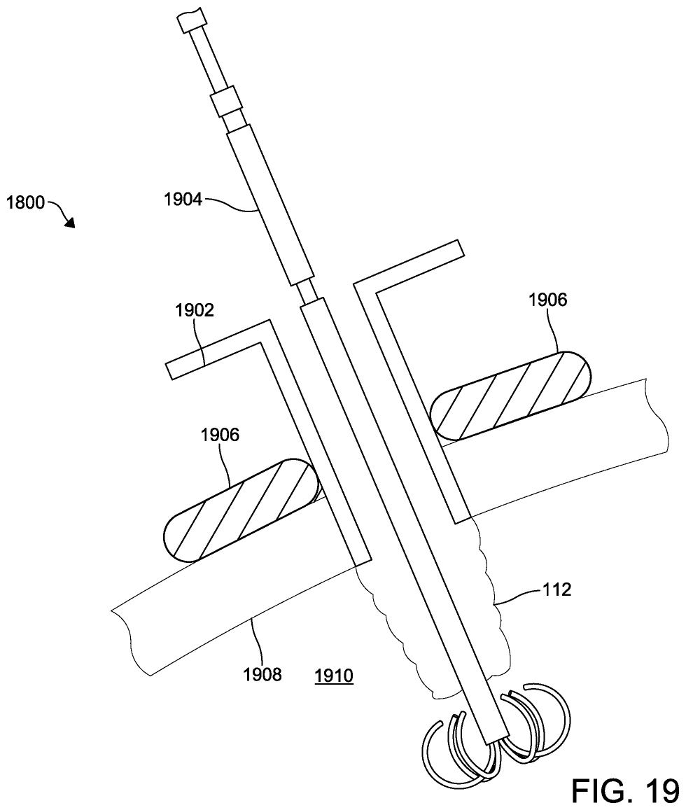

FIG. 19 shows an example system 1900. The system 1900 may comprise a port such as chest port 1902 configured to provide access, such as via a channel to a portion of a body. It should be understood that various channels or ports may be used throughout the body and the chest port 1902 is shown as a non-limiting example. As an illustrative example, the chest port 1902 is shown disposed adjacent ribs 1906 to provide access to lungs 1910 of a patient. However, other sites may be used and a chest port 1902 (or other port) may not be necessary. An anchor device 1904 may be anchored to tissue, such as the lung 1910. An example anchor device is shown in FIG. 6 for illustration. However, any suitable device for anchoring to the target site 1912 may be used. As show, the anchor device 1904 extends via the chest port 1902, through the pleura 1908, and anchors to tissue in the lung 1910. The anchor device 1904 may be anchored (e.g., releasably coupled) to a tissue at a target site 1912. The target site 1912 may comprise a core site where a portion of lung tissue has been cored, punctured, or removed. The anchor device 1904 may be placed at the target site 1912 while the lung is inflated. However, other processes may be implemented while the lung is collapsed.

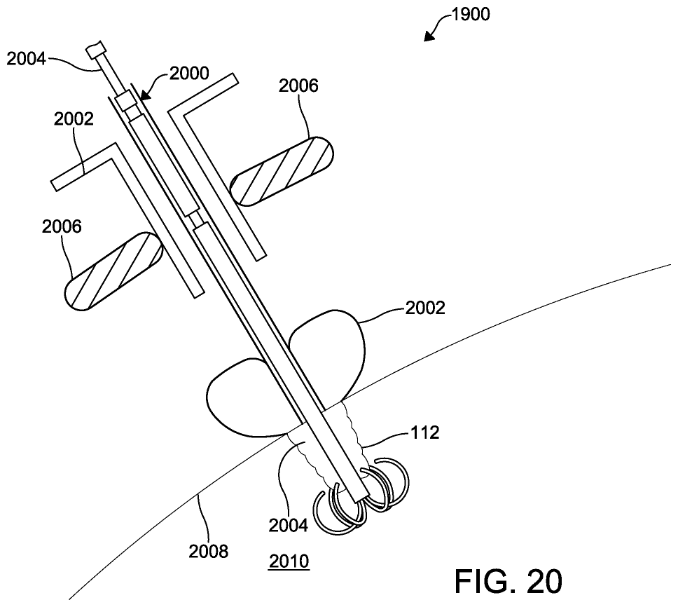

FIG. 20 shows an application of an example sealing device 2000. The sealing device 2000 may comprise an inflatable balloon 2002. Other sealing mechanisms may be used. The sealing device 2000 may comprise and/or be in contact with a balloon catheter. The balloon catheter may be a single lumen balloon catheter. The balloon catheter may be multi-lumen balloon catheter. The sealing device 2000 may be disposed adjacent the target site 2012. As such, the sealing device 2000 may seal the target site 2012 to minimize exit of a fluid or material from the target site 2012. As an example, a fill material 2004 may be disposed at the target site 2012 and may be sealed in the target site 2012 by the sealing device 2000. As an illustrative example, the inflatable balloon 2002 may provide sealing while the lung 110 moves (e.g., inflates and deflates). The sealing device 2000 may be implemented when the lung 2010 is inflated or collapsed.

Example sealing procedures are described herein and include fill materials, ablation, mechanical pressure, energy emission (e.g., RF energy), and others, for example. Causing the sealing device to seal at least a portion of the core cavity at the target site may comprise causing at least a portion of the sealing device to abut a wall defining the core cavity. Causing the sealing device to seal at least a portion of the core cavity at the target site may comprise ablating a wall defining the core cavity. Causing the sealing device to seal at least a portion of the core cavity at the target site may comprise applying pressure to a wall defining the core cavity. Methods may further comprise disposing a fill material in the core cavity, wherein the sealing device minimizes escape of the fill material from the core cavity. The fill material may comprise autologous blood. As an example, the target site may comprise at least a portion of a lung and the method may further comprise causing the lung to collapse prior to disposing the sealing device adjacent the target site. As a further example, the target site may comprise at least a portion of a lung and methods may further comprise allowing the lung to ventilate while the sealing device is sealing the target site.

An example system for implementing one or more of the methods of the present disclosure may comprise a guided anchor. The example system may comprise a single lumen balloon catheter. The example system may comprise a multi-lumen balloon catheter. The example system may comprise a coring device. Post coring by the coring device, an anchor may be introduced into the tissue cavity to ensure access to a cored site. The chest port may be removed, and the lung may be collapsed. The balloon catheter may be inserted over the anchor. Once the balloon catheter is in the chest cavity, the balloon catheter may be inflated. The inflated balloon catheter may be moved forward and pushed slightly against lung tissue. Autologous blood may be injected into a core site through the inflated balloon catheter. The inflated balloon catheter and autologous blood may be held in place for a predetermined time period (e.g., one (1) minute, etc.) to allow the blood to clot at the core site. The lung may be allowed to resume ventilation. The inflated balloon catheter may be allowed to go up and down with the lung while maintaining contact with the lung to keep the blood at the core site to facilitate further clotting. The balloon catheter may be deflated. The balloon catheter and anchor may be removed after a predetermined time period (e.g., three (3) minutes, etc.). The autologous blood may be clotted at the core site to provide pneumostasis.

In an embodiment, the anchor and/or the balloon catheter may be used to deposit autologous blood at the core site with the lung collapsed. The anchor and/or the balloon catheter may be removed right after the autologous blood is delivered. The blood may be allowed to clot in place with a predetermined time period (e.g., five (5) minutes, etc.) before the lung is allowed to resume ventilation.

The example system may cause autologous blood to be delivered to the core site. Other fill materials may be used.

The example system may allow clotted blood to provide a seal to achieve pneumostasis.

In an embodiment, a method and apparatus are provided whereby a plug or series of stitches are on a wire within the chest in a compressed configuration. When it is desired to seal the pleural space, the wire may be pulled back towards the operator, bringing the plug or stitches in opposition to the internal opening of the body space. The device may then be actuated to insert the plug or Stitches into the internal body space opening, and the wire breaks away, thereby closing the hole and preventing fluid from leaking out or air from getting sucked back in.

Polypeptide/protein-based adhesives, fibrin-based adhesives, gelatin-based adhesives, collagen-based adhesives, albumin based adhesives, polysaccharide-based adhesives, chitosan-based adhesives, human blood-based adhesives, and animal-based adhesives, and synthetic and semi-synthetic adhesives (such as cyanoacrylates, polyethylene glycol hydrogels, urethane-based adhesives, and other synthetic adhesives). The fluid may fill the volume of the tract and may be heated with RF energy or laser beyond the temperature of the surrounding tissue, to a temperature sufficient to cauterize and seal the surrounding tissue. The combination of the fluid and the RF seals the surrounding tissues

Various methods, devices, and systems may be used to core or remove tissue.

Therapy

Various therapies may be implemented.

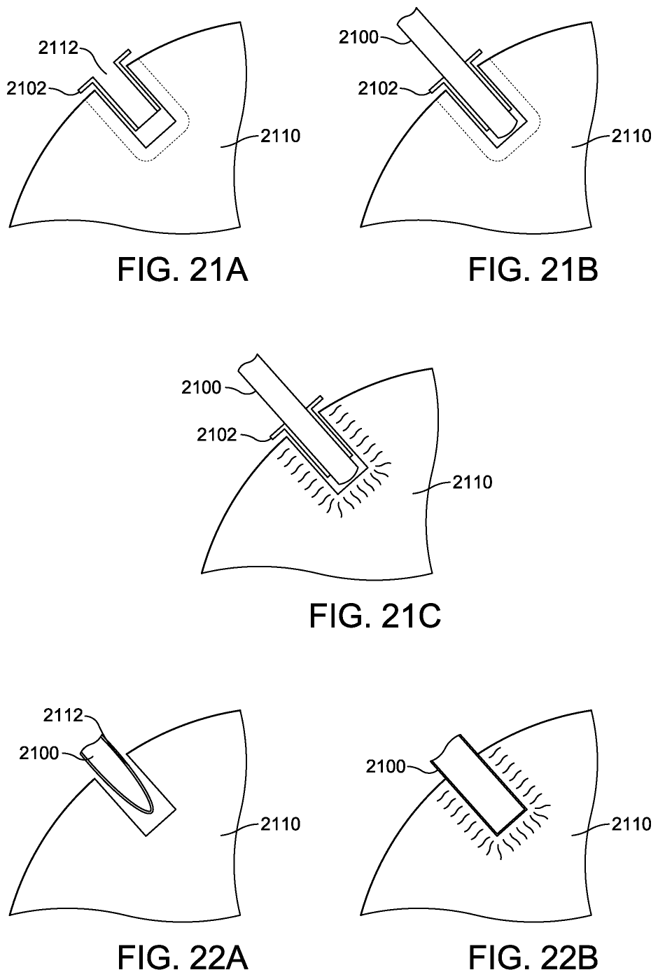

FIGS. 21-22 show illustrative examples, but other methods of ablation or energy emission may be used for sealing tissue. For example, a shaped mesh catheter may be used. As such, a catheter with collapsed meshed shape may be inserted into the cavity and the cavity sheath may be removed. The mesh may be then expanded, and suction may be applied to pull tissue to contact with the mesh. Energy, e.g. RF, may then be applied to ablate the cavity tissue wall.

Rotating Ablation Probe

FIGS. 21A-21C show an example application. As shown, once a target site has been cored out and the tissue core removed, there may be a need to ablate the tissue wall of the cavity. As such, the following ablation methods could be used. For example, a rotating ablation probe may be used. FIG. 21A shows a cored-out cavity 2112 in tissue 2110 with the cavity sheath 2102 in place to keep the cavity open. A rotating probe 2100 may then be inserted into the cavity sheath 2102, as shown in FIG. 21B. The probe 2100 may be equipped with an energy source such as an array of energy heads or a continuous energy strip. The energy may be microwave, RF, other output form. Once the probe 2100 is in place, the cavity sheath 2102 may remain in place or be removed. The energy may then be applied while the probe/energy heads are rotated to give a radially continuous ablation on the wall and bottom tissue 2110 of the cavity, as shown in FIG. 21C.

Hot Balloon Catheter

FIGS. 22A-22B show an example application. As shown, a hot balloon catheter may be used. For example, a balloon catheter 2200 may be placed into a cavity 2212 formed in tissue 2210 and a cavity sheath may be removed to expose the cavity 2212 needed to be ablated, as shown in FIG. 22A. The balloon 2200 may then be inflated with hot fluid or hot air/gas to ablate the cavity wall tissue 2210, as shown in FIG. 22B.

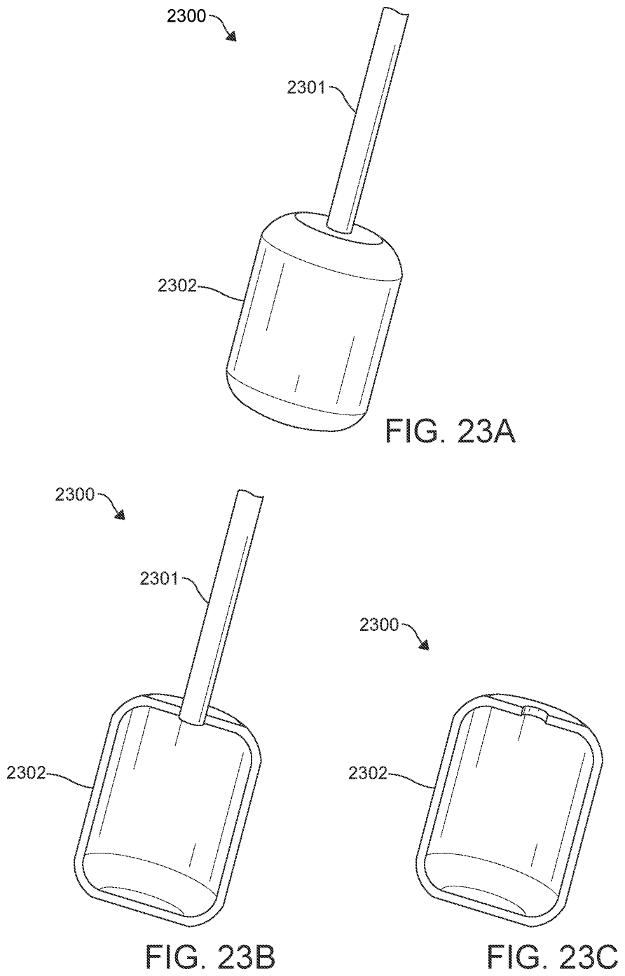

FIGS. 23A-23C show an example application. As shown, once a target site has been cored out and the tissue core removed, there may be a need to seal the cut tissue wall of the cavity. As such, the following example procedure may be used. A device 2300 may comprise a fluid conduit 2301 and an inflatable absorbable balloon 2302. The balloon 2302 may be coated on the exterior with absorbable bio adhesive that will seal against the tissue of the cored cavity post coring, as shown in FIGS. 23A-23B. Once the deflated balloon 2302 may be placed in the desired location, the balloon 2302 may be inflated with CO2 (or other fluid), for example via fluid conduit 2301, so that the bio adhesive is pressed against the tissue wall of the cored cavity to achieve sealing to prevent air leak. The CO2 filled balloon 2302 may be pressurized to an appropriate pressure and may be left behind inside the cored cavity.

When it is desired to seal the pleural space, the wire is pulled back towards the operator, bringing the plug or stitches in opposition to the internal opening of the body space.

Margin Ablation

Introducing an energy delivery device into a tissue cavity and delivering energy to eradicate cancerous tissue. Once the target tissue has been cored out and removed, the tissue wall of the cavity can be ablated. FIG. 21A shows a cored-out cavity with the cavity sheath in place to keep the cavity open. A rotating probe is then inserted into the cavity sheath as shown in FIG. 21B. The probe is equipped with an arrays of energy heads or a continuous energy strip. The energy could be microwave, RF. Once the probe is in place, the cavity sheath can remain in place or be removed. The energy is then applied while the probe/energy heads are rotated to give a radially continuous ablation on the wall and bottom tissues of the cavity, as shown in FIG. 21C.

A balloon catheter would be placed into the cavity and the cavity sheath would then be removed to expose the cavity to be ablated (FIG. 22A). The balloon is then inflated with hot fluid or hot air/gas to ablate the cavity wall tissue (FIG. 22B).

Shaped Mesh Catheter

A catheter with a collapsed meshed shape may be inserted into the cavity and the cavity sheath may be removed. The mesh may then be expanded, and suction may be applied to pull tissue to contact the mesh. Energy, e.g. RF, may then be applied to ablate the cavity tissue wall.

Microwave Ablation

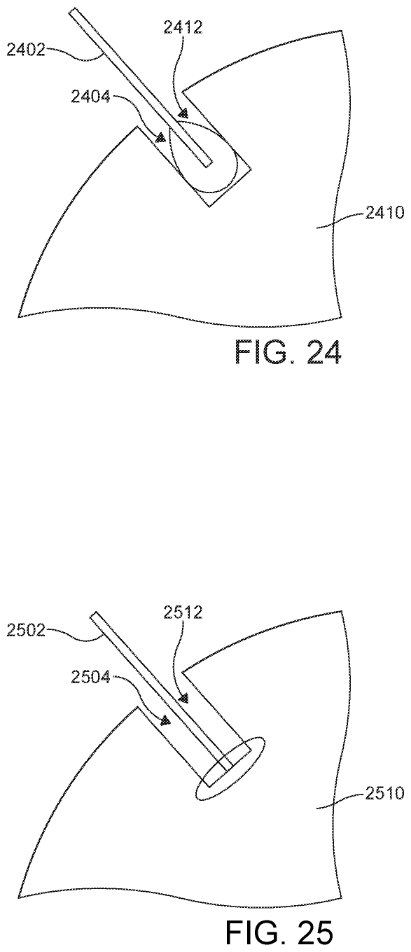

FIG. 24 illustrates an example therapy system. A catheter probe 2402 comprising an antenna 2403 which emits microwaves may be inserted into a tissue cavity 2412 cored out of tissue 2410, such as illustrated in FIG. 24. The probe 2402 (e.g., the antenna 2403) produces intense heat that ablates (e.g., destroys) the target tissue in an ablation zone 2404.

Cryoablation

FIG. 25 illustrates an example therapy system. A cryoablation probe 2502 may be inserted into a tissue cavity 2512 cored out of target tissue 2510, such as shown in FIG. 25. The probe produces extremely cold temperatures to ablate the target tissue 2510 within a cryoablation zone 2504.

Chemical Ablation (Chemoablation)

Hypertonic saline gel, solid salt, and/or acetic acid gel may be implanted into the cavity to promote damage of the target cells.

Laser Ablation (Photoablation)

A probe that emits a laser beam at a specific wavelength and pulse length may be inserted into the cavity. The emitted laser beam may be used to kill the target tissue in the cavity.

Ethanol Ablation

In this procedure, concentrated alcohol in liquid or gel form may be injected directly into the target cavity to damage the cells.

Chemotherapy Drugs

At the cored site, administration of chemotherapy drugs such as doxorubicin, fluorouracil, and/or cisplatin may be done via direct injection of the agent into the cored tissue site.

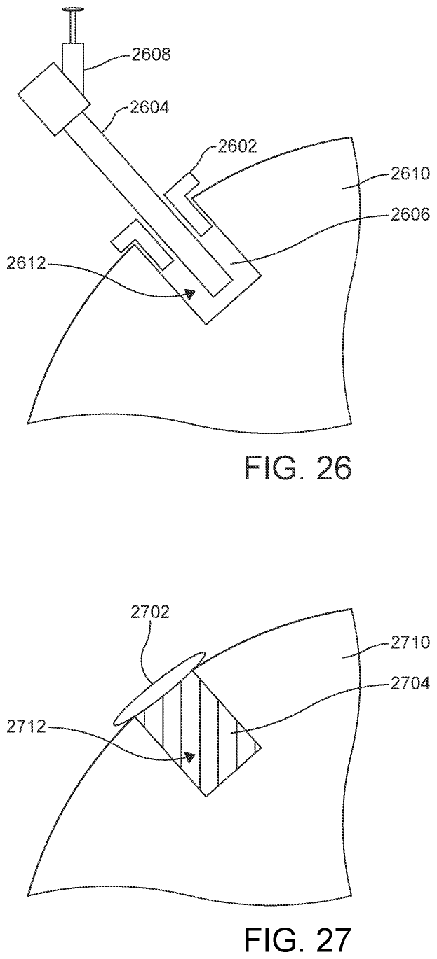

FIG. 26 illustrates an example therapy system comprising a delivery probe 2600. The method of drug/therapy delivery may be achieved by placing a cavity sheath 2602 into a tissue cavity 2612 at the cored site of cored tissue 2610. Then, a delivery probe 2604 containing one or more lumens 2606 at the distal end may be inserted into the cavity sheath 2602. Said delivery probe 2604 may extend out of the distal opening of said cavity sheath 2602 into the cored tissue cavity 2612. The desired therapeutic and/or diagnostic agent may then be delivered through the delivery lumen 2606 to the tissue via the distal end of the delivery probe via direct injection using a drug/therapy injection port with plunger 2608, such as shown in FIG. 26.

FIG. 27 illustrates an example therapy system. In some scenarios, a biodegradable plug 2702 may be placed over a site of cored tissue 2710 following the addition of the drug/therapy to the cavity, such as shown in FIGS. 26-27. Namely, drug 2704 may be delivered into a tissue cavity 2712 at the cored site of cored tissue 2710. The plug 2702 may be secured in place using a biocompatible glue. The plug 2702 may be configured to minimize material from exiting the tissue cavity 2712 such as the drug 2704.

Chemotherapy Drug-Eluting Particles

Chemotherapy drug-eluting particles may be delivered to the cored tissue site, thereby promoting controlled and sustained locoregional release of therapeutic agents in high concentration with prolonged administration. For example, doxorubicin may be encapsulated into nanoparticles to form micelles for targeted drug delivery. Additionally, anti-cancer drugs may be vectorized using porous particles, such as mesoporous silica nanoparticles, and delivered to the cored tissue site.

Co-Delivery of siRNA and Chemotherapy Drugs

Chemotherapy drugs and short interfering RNA (siRNA) may be co-delivered to the cored tissue site through direct injection to promote cancer cell death. Multidrug resistance in cancer cells may be suppressed using siRNA-based formulations to induce specific silencing of a broad range of genetic targets. Delivering siRNAs in combination with chemotherapy drugs may enhance the efficacy of the chemotherapy through conquering the resistance mechanism of the cancer cells. For example, siRNA encapsulated in mesoporous silica nanoparticles may be co-delivered with doxorubicin to the target core site.

Biodegradable Hydrogel-Based Controlled Drug Delivery

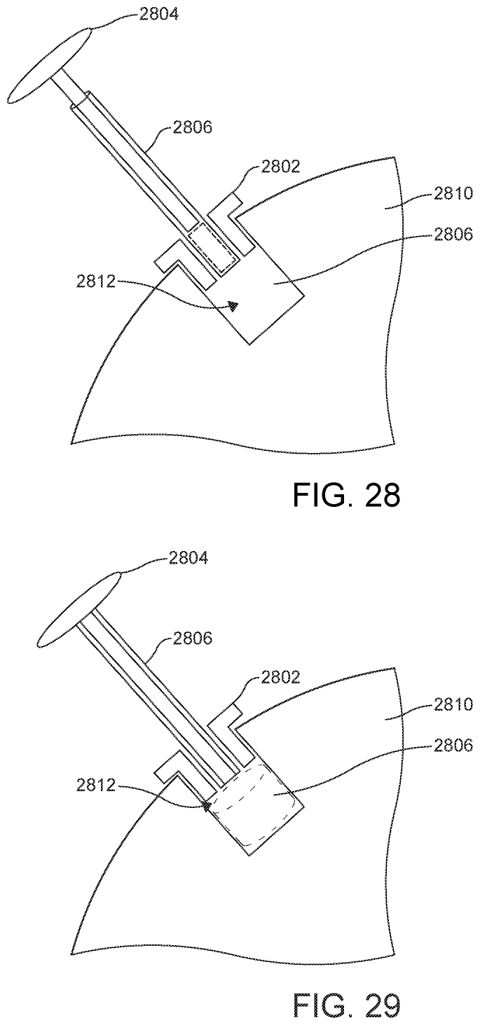

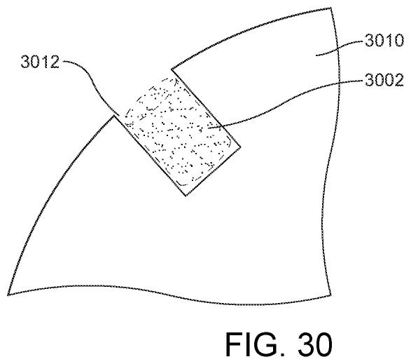

FIGS. 28-29 illustrate an example therapy system and method. The method of hydrogel/plug delivery may be achieved by placing a cavity sheath 2802 into a tissue cavity 2812 at the cored site 2810. Then, a delivery plunger 2804, further comprising a delivery plunger sheath 2806, and containing a hydrogel 2807 at the distal end 2814 may be inserted through the cavity sheath 2802 into the cored site 2812. The hydrogel 2807 may then be delivered into the cored site 2812 through the plunging mechanism of the delivery plunger 2802, such as is shown in FIGS. 28-29.

Photodynamic Therapy (PDT)

A combination of chemotherapy drug(s) and photodynamic therapy (PDT) may be directly delivered to the cored tissue site. PDT is a treatment modality which relies on a photosensitizer and light to generate reactive oxygen species (ROS) to kill cancer cells.

Degradable Polymer/Scaffold System

FIG. 30 illustrates an example therapy system. Polymer systems containing chemotherapy drugs may be delivered to the cored tissue site 3012 of cored tissue 3010 via direct implantation. Porous biodegradable polymers, such as sponges or scaffolds 3002, may be designed to carry chemotherapy drugs, such as cisplatin. These polymers degrade overtime, thereby releasing the chemotherapy drug at a controlled rate within the targeted site. The excellent biodegradability of the scaffolds, such as porous scaffolds, overcome the limitations of non-biodegradable systems which support the sustained release of the chemotherapy drugs and degrade after a specific time period. The scaffold 3002 may be manufactured in manner that is convenient for surgical delivery, such as shown in FIG. 30.

Hyperthermia of Cored Tissue Site

Hyperthermia may be used to treat the desired cored tissue site. Using this approach, the cored tissue site may be exposed to higher than normal temperatures to promote selective destruction of abnormal cells, which minimizes the size effects on healthy cells. For example, light-absorbing metal particles, such as gold nanoparticles or iron oxide microparticles, may be delivered to the cored tissue site. Then, by applying a short-pulsed laser, cancer cells targeted with the metal particles may be killed.

The present disclosure comprises at least the following aspects:

Aspect 1. A method for coring tissue, the method comprising: determining a location of a tissue lesion at a tissue site of a body; navigating an instrument based on a location of the tissue lesion; coupling the instrument to the tissue lesion or adjacent the tissue lesion; configuring access to the tissue site; introducing a tissue resection device to the tissue site; using the tissue resection device to create a core of tissue; removing the core of tissue from the body to create a tissue cavity; analyzing the tissue core sample; sealing the tissue cavity; removing some or all instrumentation; and closing tissue access points.

Aspect 2. The method of aspect 1, wherein the tissue site comprise lung tissue.

Aspect 3. The method of any one of aspects 1-2, wherein the navigating the instrument defines a pathway and wherein the introducing the tissue resection device is based on the pathway.

Aspect 4. The method of any one of aspects 1-3, wherein the introducing the tissue resection device is implemented using at least a portion of the instrument.

Aspect 5. The method of any one of aspects 1-4, wherein the core of tissue comprises at least a portion of the tissue lesion.

Aspect 6. The method of any one of aspects 1-5, wherein the determining the location of a tissue lesion comprises using an imaging system.

Aspect 7. The method of aspect 6, wherein the imaging system comprises one or more of computer tomography, ultrasound, magnetic resonance imaging, endoscope, visual, electromagnetic, or X-ray.

Aspect 8. The method of any one of aspects 1-7, wherein the navigating an instrument to the tissue lesion comprising using a robotic system.

Aspect 9. The method of any one of aspects 1-8, wherein the navigating an instrument to the tissue lesion comprising using one or more of computer tomography, ultrasound, electromagnetic navigation, or fluoroscopic navigation.

Aspect 10. The method of any one of aspects 1-9, wherein the configuring access to the tissue site comprises using chest wall incision blades.

Aspect 11. The method of any one of aspects 1-10, wherein the configuring access to the tissue site comprises using tissue dilation.

Aspect 12. The method of any one of aspects 1-11, wherein the configuring access to the tissue site comprises using a trocar.

Aspect 13. The method of any one of aspects 1-12, wherein the configuring access to the tissue site comprises using an open incision.

Aspect 14. The method of any one of aspects 1-13, wherein the analyzing the tissue core sample comprises using rapid on-site evaluation.

Aspect 15. The method of any one of aspects 1-14, wherein the analyzing the tissue core sample comprises using histology.

Aspect 16. The method of any one of aspects 1-15, wherein the analyzing the tissue core sample comprises using immunohistochemistry.

Aspect 17. The method of any one of aspects 1-16, wherein the analyzing the tissue core sample comprises using electron microscopy.

Aspect 18. The method of any one of aspects 1-17, wherein the analyzing the tissue core sample comprises using flow cytometry.

Aspect 19. The method of any one of aspects 1-18, wherein the analyzing the tissue core sample comprises using image cytometry.

Aspect 20. The method of any one of aspects 1-19, wherein the analyzing the tissue core sample comprises using polymerase chain reaction.

Aspect 21. The method of any one of aspects 1-20, wherein the analyzing the tissue core sample comprises using gene expression microarrays.

Aspect 22. The method of any one of aspects 1-21, wherein the analyzing the tissue core sample comprises using fluorescent in situ hybridization.

Aspect 23. The method of any one of aspects 1-22, wherein the analyzing the tissue core sample comprises using genetic sequencing.

Aspect 24. The method of any one of aspects 1-23, wherein the analyzing the tissue core sample comprises using atomic force microscopy.

Aspect 25. The method of any one of aspects 1-24, wherein the analyzing the tissue core sample comprises using surface enhanced Ramen spectroscopy.

Aspect 26. A method for coring tissue, the method comprising: determining a location of a tissue lesion at a tissue site of a body; navigating an instrument based on a location of the tissue lesion; coupling the instrument to the tissue lesion or adjacent the tissue lesion; introducing a tissue resection device to the tissue site; using the tissue resection device to create a core of tissue; removing the core of tissue from the body to create a tissue cavity; analyzing the tissue core sample; sealing the tissue cavity; and removing some or all instrumentation.

Aspect 27. The method of aspect 26, wherein the tissue site comprise lung tissue.

Aspect 28. The method of any one of aspects 26-27, wherein the navigating the instrument defines a pathway and wherein the introducing the tissue resection device is based on the pathway.

Aspect 29. The method of any one of aspects 26-28, wherein the introducing the tissue resection device is implemented using at least a portion of the instrument.

Aspect 30. The method of any one of aspects 26-29, wherein the core of tissue comprises at least a portion of the tissue lesion.

Aspect 31. The method of any one of aspects 26-30, wherein the determining the location of a tissue lesion comprises using an imaging system.

Aspect 32. The method of aspect 31, wherein the imaging system comprises one or more of computer tomography, ultrasound, magnetic resonance imaging, endoscope, visual, electromagnetic, or X-ray.

Aspect 33. The method of any one of aspects 26-32, wherein the navigating an instrument to the tissue lesion comprising using a robotic system.

Aspect 34. The method of any one of aspects 26-33, wherein the navigating an instrument to the tissue lesion comprising using one or more of computer tomography, ultrasound, electromagnetic navigation, or fluoroscopic navigation.

Aspect 35. The method of any one of aspects 26-34, further comprising configuring access to the tissue site wherein the configuring access to the tissue site comprises using chest wall incision blades.

Aspect 36. The method of any one of aspects 26-35, further comprising configuring access to the tissue site wherein the configuring access to the tissue site comprises using tissue dilation.

Aspect 37. The method of any one of aspects 26-36, further comprising configuring access to the tissue site wherein the configuring access to the tissue site comprises using a trocar.

Aspect 38. The method of any one of aspects 26-37, further comprising configuring access to the tissue site wherein the configuring access to the tissue site comprises using an open incision.

Aspect 39. The method of any one of aspects 26-38, wherein the analyzing the tissue core sample comprises using rapid on-site evaluation.

Aspect 40. The method of any one of aspects 26-39, wherein the analyzing the tissue core sample comprises using histology.

Aspect 41. The method of any one of aspects 26-40, wherein the analyzing the tissue core sample comprises using immunohistochemistry.

Aspect 42. The method of any one of aspects 26-41, wherein the analyzing the tissue core sample comprises using electron microscopy.

Aspect 43. The method of any one of aspects 26-42, wherein the analyzing the tissue core sample comprises using flow cytometry.

Aspect 44. The method of any one of aspects 26-43, wherein the analyzing the tissue core sample comprises using image cytometry.

Aspect 45. The method of any one of aspects 26-44, wherein the analyzing the tissue core sample comprises using polymerase chain reaction.

Aspect 46. The method of any one of aspects 26-45, wherein the analyzing the tissue core sample comprises using gene expression microarrays.

Aspect 47. The method of any one of aspects 26-46, wherein the analyzing the tissue core sample comprises using fluorescent in situ hybridization.

Aspect 48. The method of any one of aspects 26-47, wherein the analyzing the tissue core sample comprises using genetic sequencing.

Aspect 49. The method of any one of aspects 26-48, wherein the analyzing the tissue core sample comprises using atomic force microscopy.

Aspect 50. The method of any one of aspects 26-49, wherein the analyzing the tissue core sample comprises using surface enhanced Ramen spectroscopy.

Aspect 51. A method for coring tissue, the method comprising: determining the location of a tissue lesion; navigating an instrument based on a location of the tissue lesion; coupling the instrument to the tissue lesion or adjacent the tissue lesion; configuring access to the tissue site; introducing a tissue resection device to the tissue site; using the tissue resection device to create a core of tissue; removing the core of tissue from the body; analyzing the tissue core sample; performing a diagnostic procedure using at least the tissue core sample; sealing the tissue cavity; removing some or all instrumentation; and closing tissue access points.

Aspect 52. The method of aspect 51, wherein the core of tissue comprises at least a portion of the tissue lesion.

Aspect 53. The method of any one of aspects 51-52, wherein the determining the location of a tissue lesion comprises using an imaging system.

Aspect 54. The method of aspect 53, wherein the imaging system comprises one or more of computer tomography, ultrasound, magnetic resonance imaging, endoscope, visual, electromagnetic, or X-ray.

Aspect 55. The method of any one of aspects 51-54, wherein the navigating an instrument to the tissue lesion comprising using a robotic system.

Aspect 56. The method of any one of aspects 51-55, wherein the navigating an instrument to the tissue lesion comprising using one or more of computer tomography, ultrasound, electromagnetic navigation, or fluoroscopic navigation.

Aspect 57. The method of any one of aspects 51-56, wherein the configuring access to the tissue site comprises using chest wall incision blades.

Aspect 58. The method of any one of aspects 51-57, wherein the configuring access to the tissue site comprises using tissue dilation.

Aspect 59. The method of any one of aspects 51-58, wherein the configuring access to the tissue site comprises using a trocar.

Aspect 60. The method of any one of aspects 51-59, wherein the configuring access to the tissue site comprises using an open incision.

Aspect 61. The method of any one of aspects 51-60, wherein the analyzing the tissue core sample comprises using rapid on-site evaluation.

Aspect 62. The method of any one of aspects 51-61, wherein the analyzing the tissue core sample comprises using histology.

Aspect 63. The method of any one of aspects 51-62, wherein the analyzing the tissue core sample comprises using immunohistochemistry.

Aspect 64. The method of any one of aspects 51-63, wherein the analyzing the tissue core sample comprises using electron microscopy.

Aspect 65. The method of any one of aspects 51-64, wherein the analyzing the tissue core sample comprises using flow cytometry.

Aspect 66. The method of any one of aspects 51-65, wherein the analyzing the tissue core sample comprises using image cytometry.

Aspect 67. The method of any one of aspects 51-66, wherein the analyzing the tissue core sample comprises using polymerase chain reaction.

Aspect 68. The method of any one of aspects 51-67, wherein the analyzing the tissue core sample comprises using gene expression microarrays.

Aspect 69. The method of any one of aspects 51-68, wherein the analyzing the tissue core sample comprises using fluorescent in situ hybridization.

Aspect 70. The method of any one of aspects 51-69, wherein the analyzing the tissue core sample comprises using genetic sequencing.

Aspect 71. The method of any one of aspects 51-70, wherein the analyzing the tissue core sample comprises using atomic force microscopy.

Aspect 72. The method of any one of aspects 51-71, wherein the analyzing the tissue core sample comprises using surface enhanced Ramen spectroscopy.

Aspect 73. A method for coring tissue, the method comprising: determining the location of a tissue lesion; navigating an instrument based on a location of the tissue lesion; coupling the instrument to the tissue lesion or adjacent the tissue lesion; configuring access to the tissue site; introducing a tissue resection device to the tissue site; using the tissue resection device to create a core of tissue; removing the core of tissue from the body; analyzing the tissue core sample; performing therapeutic management of tissue; sealing the tissue cavity; removing some or all instrumentation; and closing tissue access points.

Aspect 74. The method of aspect 73, wherein the core of tissue comprises at least a portion of the tissue lesion.

Aspect 75. The method of any one of aspects 73-74, wherein the determining the location of a tissue lesion comprises using an imaging system.

Aspect 76. The method of aspect 75, wherein the imaging system comprises one or more of computer tomography, ultrasound, magnetic resonance imaging, endoscope, visual, electromagnetic, or X-ray.

Aspect 77. The method of any one of aspects 73-76, wherein the navigating an instrument to the tissue lesion comprising using a robotic system.

Aspect 78. The method of any one of aspects 73-77, wherein the navigating an instrument to the tissue lesion comprising using one or more of computer tomography, ultrasound, electromagnetic navigation, or fluoroscopic navigation.

Aspect 79. The method of any one of aspects 73-78, wherein the configuring access to the tissue site comprises using chest wall incision blades.

Aspect 80. The method of any one of aspects 73-79, wherein the configuring access to the tissue site comprises using tissue dilation.

Aspect 81. The method of any one of aspects 73-80, wherein the configuring access to the tissue site comprises using a trocar.

Aspect 82. The method of any one of aspects 73-81, wherein the configuring access to the tissue site comprises using an open incision.

Aspect 83. The method of any one of aspects 73-82, wherein the analyzing the tissue core sample comprises using rapid on-site evaluation.

Aspect 84. The method of any one of aspects 73-83, wherein the analyzing the tissue core sample comprises using histology.

Aspect 85. The method of any one of aspects 73-84, wherein the analyzing the tissue core sample comprises using immunohistochemistry.

Aspect 86. The method of any one of aspects 73-85, wherein the analyzing the tissue core sample comprises using electron microscopy.

Aspect 87. The method of any one of aspects 73-86, wherein the analyzing the tissue core sample comprises using flow cytometry.

Aspect 88. The method of any one of aspects 73-87, wherein the analyzing the tissue core sample comprises using image cytometry.

Aspect 89. The method of any one of aspects 73-88, wherein the analyzing the tissue core sample comprises using polymerase chain reaction.

Aspect 90. The method of any one of aspects 73-89, wherein the analyzing the tissue core sample comprises using gene expression microarrays.

Aspect 91. The method of any one of aspects 73-90, wherein the analyzing the tissue core sample comprises using fluorescent in situ hybridization.

Aspect 92. The method of any one of aspects 73-91, wherein the analyzing the tissue core sample comprises using genetic sequencing.

Aspect 93. The method of any one of aspects 73-92, wherein the analyzing the tissue core sample comprises using atomic force microscopy.

Aspect 94. The method of any one of aspects 73-93, wherein the analyzing the tissue core sample comprises using surface enhanced Ramen spectroscopy.

Aspect 95. The method of any one of aspects 73-94, wherein the performing therapeutic management of tissue comprises ablation.

Aspect 96. The method of any one of aspects 73-95, wherein the performing therapeutic management of tissue comprises chemotherapy.

Aspect 97. The method of any one of aspects 73-96, wherein the performing therapeutic management of tissue comprises hydrogel-based controlled drug delivery.

Aspect 98. The method of any one of aspects 73-97, wherein the performing therapeutic management of tissue comprises photodynamic therapy.

Aspect 99. The method of any one of aspects 73-98, wherein the performing therapeutic management of tissue comprises using a scaffold system.

Aspect 100. The method of any one of aspects 73-99, wherein the performing therapeutic management of tissue comprises hyperthermia of cored tissue site.

Aspect 101. The method of any one of aspects 73-100, wherein the performing therapeutic management of tissue is based on the analyzing the tissue core sample.

Aspect 102. The method of any one of aspects 73-101, wherein the performing therapeutic management of tissue is implemented on benign tissue.

Aspect 103. The method of any one of aspects 73-102, wherein the performing therapeutic management of tissue is implemented on malignant tissue.

Aspect 104. A method for coring tissue, the method comprising: introducing a tissue resection device to the tissue site; using the tissue resection device to create a core of tissue; removing at least a portion of the core of tissue from the body; analyzing the tissue core sample; and sealing the tissue cavity.

Aspect 105. The method of aspect 104, further comprising using an imaging system to determine the location of a tissue lesion within the target tissue site.

Aspect 106. The method of aspect 105, wherein the imaging system comprises one or more of computer tomography, ultrasound, magnetic resonance imaging, endoscope, visual, electromagnetic, or X-ray.

Aspect 107. The method of any one of aspects 104-106, further comprising using a robotic system to navigate an instrument to the target tissue site.

Aspect 108. The method of any one of aspects 104-107, further comprising using one or more of computer tomography, ultrasound, electromagnetic navigation, or fluoroscopic navigation to navigate an instrument to the target tissue site.

Aspect 109. The method of any one of aspects 104-108, wherein the analyzing the tissue core sample comprises using rapid on-site evaluation.

Aspect 110. The method of any one of aspects 104-109, wherein the analyzing the tissue core sample comprises using histology.

Aspect 111. The method of any one of aspects 104-110, wherein the analyzing the tissue core sample comprises using immunohistochemistry.

Aspect 112. The method of any one of aspects 104-111, wherein the analyzing the tissue core sample comprises using electron microscopy.

Aspect 113. The method of any one of aspects 104-112, wherein the analyzing the tissue core sample comprises using flow cytometry.

Aspect 114. The method of any one of aspects 104-113, wherein the analyzing the tissue core sample comprises using image cytometry.

Aspect 115. The method of any one of aspects 104-114, wherein the analyzing the tissue core sample comprises using polymerase chain reaction.

Aspect 116. The method of any one of aspects 104-115, wherein the analyzing the tissue core sample comprises using gene expression microarrays.

Aspect 117. The method of any one of aspects 104-116, wherein the analyzing the tissue core sample comprises using fluorescent in situ hybridization.

Aspect 118. The method of any one of aspects 104-117, wherein the analyzing the tissue core sample comprises using genetic sequencing.

Aspect 119. The method of any one of aspects 104-118, wherein the analyzing the tissue core sample comprises using atomic force microscopy.

Aspect 120. The method of any one of aspects 104-119, wherein the analyzing the tissue core sample comprises using surface enhanced Ramen spectroscopy.

Aspect 121. The method of any one of aspects 104-120, further comprising determining a diagnostic using at least the tissue core sample.

Aspect 122. The method of any one of aspects 104-121, further comprising performing therapeutic management of tissue.

Aspect 123. The method of any one of aspects 104-122, further comprising performing therapeutic management of benign tissue.

Aspect 124. The method of any one of aspects 104-123, further comprising performing therapeutic management of malignant tissue.

Aspect 125. The method of aspect 124, wherein the performing therapeutic management of malignant tissue comprises ablation.

Aspect 126. The method of any one of aspects 104-124, wherein the performing therapeutic management of malignant tissue comprises chemotherapy.

Aspect 127. The method of any one of aspects 104-124, wherein the performing therapeutic management of malignant tissue comprises hydrogel-based controlled drug delivery.

Aspect 128. The method of any one of aspects 104-124, wherein the performing therapeutic management of malignant tissue comprises photodynamic therapy.

Aspect 129. The method of any one of aspects 104-124, wherein the performing therapeutic management of malignant tissue comprises using a scaffold system.

Aspect 130. The method of any one of aspects 104-124, wherein the performing therapeutic management of malignant tissue comprises hyperthermia of cored tissue site.

Aspect 131. A method for coring tissue, the method comprising: determining the location of a tissue lesion; navigating an instrument based on a location of the tissue lesion; coupling the instrument to the tissue lesion or adjacent the tissue lesion; introducing a tissue resection device to the tissue site; using the tissue resection device to create a core of tissue; removing the core of tissue from the body; analyzing the tissue core sample; performing therapeutic management of tissue; sealing the tissue cavity; and removing some or all instrumentation.

Aspect 132. The method of aspect 131, wherein the core of tissue comprises at least a portion of the tissue lesion.

Aspect 133. The method of any one of aspects 131-132, wherein the determining the location of a tissue lesion comprises using an imaging system.

Aspect 134. The method of aspect 133, wherein the imaging system comprises one or more of computer tomography, ultrasound, magnetic resonance imaging, endoscope, visual, electromagnetic, or X-ray.

Aspect 135. The method of any one of aspects 131-133, wherein the navigating an instrument to the tissue lesion comprising using a robotic system.

Aspect 136. The method of any one of aspects 131-135, wherein the navigating an instrument to the tissue lesion comprising using one or more of computer tomography, ultrasound, electromagnetic navigation, or fluoroscopic navigation.

Aspect 137. The method of any one of aspects 131-136, further comprising configuring access to the tissue site wherein the configuring access to the tissue site comprises using chest wall incision blades.

Aspect 138. The method of any one of aspects 131-137, further comprising configuring access to the tissue site wherein the configuring access to the tissue site comprises using tissue dilation.

Aspect 139. The method of any one of aspects 131-138, further comprising configuring access to the tissue site wherein the configuring access to the tissue site comprises using a trocar.

Aspect 140. The method of any one of aspects 131-139, further comprising configuring access to the tissue site wherein the configuring access to the tissue site comprises using an open incision.

Aspect 141. The method of any one of aspects 131-140, wherein the analyzing the tissue core sample comprises using rapid on-site evaluation.

Aspect 142. The method of any one of aspects 131-141, wherein the analyzing the tissue core sample comprises using histology.

Aspect 143. The method of any one of aspects 131-142, wherein the analyzing the tissue core sample comprises using immunohistochemistry.

Aspect 144. The method of any one of aspects 131-143, wherein the analyzing the tissue core sample comprises using electron microscopy.

Aspect 145. The method of any one of aspects 131-144, wherein the analyzing the tissue core sample comprises using flow cytometry.

Aspect 146. The method of any one of aspects 131-145, wherein the analyzing the tissue core sample comprises using image cytometry.

Aspect 147. The method of any one of aspects 131-146, wherein the analyzing the tissue core sample comprises using polymerase chain reaction.

Aspect 148. The method of any one of aspects 131-147, wherein the analyzing the tissue core sample comprises using gene expression microarrays.

Aspect 149. The method of any one of aspects 131-148, wherein the analyzing the tissue core sample comprises using fluorescent in situ hybridization.

Aspect 150. The method of any one of aspects 131-149, wherein the analyzing the tissue core sample comprises using genetic sequencing.

Aspect 151. The method of any one of aspects 131-150, wherein the analyzing the tissue core sample comprises using atomic force microscopy.

Aspect 152. The method of any one of aspects 131-151, wherein the analyzing the tissue core sample comprises using surface enhanced Ramen spectroscopy.

Aspect 153. The method of any one of aspects 131-152, wherein the performing therapeutic management of tissue comprises ablation.

Aspect 154. The method of any one of aspects 131-153, wherein the performing therapeutic management of tissue comprises chemotherapy.

Aspect 155. The method of any one of aspects 131-154, wherein the performing therapeutic management of tissue comprises hydrogel-based controlled drug delivery.

Aspect 156. The method of any one of aspects 131-155, wherein the performing therapeutic management of tissue comprises photodynamic therapy.

Aspect 157. The method of any one of aspects 131-156, wherein the performing therapeutic management of tissue comprises using a scaffold system.

Aspect 158. The method of any one of aspects 131-157, wherein the performing therapeutic management of tissue comprises hyperthermia of cored tissue site.

Aspect 159. The method of any one of aspects 131-158, wherein the performing therapeutic management of tissue is based on the analyzing the tissue core sample.

Aspect 160. The method of any one of aspects 131-159, wherein the performing therapeutic management of tissue is implemented on benign tissue.

Aspect 161. The method of any one of aspects 131-160, wherein the performing therapeutic management of tissue is implemented on malignant tissue.

Although shown and described is what is believed to be the most practical and preferred embodiments, it is apparent that departures from specific designs and methods described and shown will suggest themselves to those skilled in the art and may be used without departing from the spirit and scope of the invention. For example, the systems, devices and methods described herein for removal of lesions from the lung. It will be appreciated by the skilled artisan that the devices and methods described herein may are not limited to the lung and could be used for tissue resection and lesion removal in other areas of the body. The present invention is not restricted to the particular constructions described and illustrated, but should be constructed to cohere with all modifications that may fall within the scope of the appended claims.

What is claimed is:

1. A method for coring tissue, the method comprising:
determining a location of a tissue lesion at a tissue site of a body;
navigating an instrument based on a location of the tissue lesion;
coupling the instrument to the tissue lesion or adjacent the tissue lesion;
configuring access to the tissue site;
introducing a tissue resection device to the tissue site;
using the tissue resection device to create a core of tissue;
removing the core of tissue from the body to create a tissue cavity;
analyzing the tissue core sample;
sealing the tissue cavity;
removing some or all instrumentation; and
closing tissue access points.

2. The method of claim 1, wherein the tissue site comprise lung tissue.

3. The method of claim 1, wherein the navigating the instrument defines a pathway and wherein the introducing the tissue resection device is based on the pathway.

4. The method of claim 1, wherein the introducing the tissue resection device is implemented using at least a portion of the instrument.

5. The method of claim 1, wherein the core of tissue comprises at least a portion of the tissue lesion.

6. The method of claim 1, wherein the determining the location of a tissue lesion comprises using an imaging system.

7. The method of claim 6, wherein the imaging system comprises one or more of computer tomography, ultrasound, magnetic resonance imaging, endoscope, visual, electromagnetic, or X-ray.

8. The method of claim 1, wherein the navigating an instrument to the tissue lesion comprising using a robotic system.

9. The method of claim 1, wherein the navigating an instrument to the tissue lesion comprising using one or more of computer tomography, ultrasound, electromagnetic navigation, or fluoroscopic navigation.

10. The method of claim 1, wherein the configuring access to the tissue site comprises using one or more of chest wall incision blades, tissue dilation, a trocar, or an open incision.

11. The method of claim 1, wherein the analyzing the tissue core sample comprises using one or more of rapid on-site evaluation, histology, immunohistochemistry, electron microscopy, flow cytometry, image cytometry, polymerase chain reaction, gene expression microarrays, fluorescent in situ hybridization, genetic sequencing, atomic force microscopy, or surface enhanced Ramen spectroscopy.

12. A method for coring tissue, the method comprising:
determining a location of a tissue lesion at a tissue site of a body;
navigating an instrument based on a location of the tissue lesion;
coupling the instrument to the tissue lesion or adjacent the tissue lesion;
introducing a tissue resection device to the tissue site;
using the tissue resection device to create a core of tissue;
removing the core of tissue from the body to create a tissue cavity;
analyzing the tissue core sample;
sealing the tissue cavity; and
removing some or all instrumentation.

13. The method of claim 12, wherein the tissue site comprise lung tissue.

14. The method of claim 12, wherein the navigating the instrument defines a pathway and wherein the introducing the tissue resection device is based on the pathway.

15. The method of claim 12, wherein the introducing the tissue resection device is implemented using at least a portion of the instrument.

16. The method of claim 12, wherein the core of tissue comprises at least a portion of the tissue lesion.

17. The method of claim 12, wherein the determining the location of a tissue lesion comprises using an imaging system.

18. The method of claim 17, wherein the imaging system comprises one or more of computer tomography, ultrasound, magnetic resonance imaging, endoscope, visual, electromagnetic, or X-ray.

19. The method of claim 12, wherein the navigating an instrument to the tissue lesion comprising using a robotic system.

20. The method of claim 12, wherein the navigating an instrument to the tissue lesion comprising using one or more of computer tomography, ultrasound, electromagnetic navigation, or fluoroscopic navigation.

21. The method of claim 12, further comprising configuring access to the tissue site wherein the configuring access to the tissue site comprises using one or more of one or more of chest wall incision blades, tissue dilation, a trocar, or an open incision.

22. The method of claim 12, wherein the analyzing the tissue core sample comprises using one or more of rapid on-site evaluation, histology, immunohistochemistry, electron microscopy, flow cytometry, image cytometry, polymerase chain reaction, gene expression microarrays, fluorescent in situ hybridization, genetic sequencing, atomic force microscopy, or surface enhanced Ramen spectroscopy.

23. A method for coring tissue, the method comprising:
determining the location of a tissue lesion;
navigating an instrument based on a location of the tissue lesion;
coupling the instrument to the tissue lesion or adjacent the tissue lesion;
configuring access to a tissue site comprising the tissue lesion;
introducing a tissue resection device to the tissue site;
using the tissue resection device to create a core of tissue;
removing the core of tissue from the body, thereby defining at least a portion of a tissue cavity;
analyzing the tissue core sample;
performing a diagnostic procedure using at least the tissue core sample;
sealing the tissue cavity;
removing some or all instrumentation; and
closing tissue access points.

24. The method of claim 23, wherein the core of tissue comprises at least a portion of the tissue lesion.

25. The method of claim 23, wherein the determining the location of a tissue lesion comprises using an imaging system.

26. The method of claim 25, wherein the imaging system comprises one or more of computer tomography, ultrasound, magnetic resonance imaging, endoscope, visual, electromagnetic, or X-ray.

27. The method of claim 23, wherein the navigating an instrument to the tissue lesion comprising using a robotic system.

28. The method of claim 23, wherein the navigating an instrument to the tissue lesion comprising using one or more of computer tomography, ultrasound, electromagnetic navigation, or fluoroscopic navigation.

29. The method of claim 23, wherein the configuring access to the tissue site comprises using one or more of one or more of chest wall incision blades, tissue dilation, a trocar, or an open incision.

30. The method of claim 23, wherein the analyzing the tissue core sample comprises using one or more of rapid on-site evaluation, histology, immunohistochemistry, electron microscopy, flow cytometry, image cytometry, polymerase chain reaction, gene expression microarrays, fluorescent in situ hybridization, genetic sequencing, atomic force microscopy, or surface enhanced Ramen spectroscopy.

31. A method for coring tissue, the method comprising:
determining the location of a tissue lesion;
navigating an instrument based on a location of the tissue lesion;
coupling the instrument to the tissue lesion or adjacent the tissue lesion;
configuring access to a tissue site comprising the tissue lesion;
introducing a tissue resection device to the tissue site;
using the tissue resection device to create a core of tissue;
removing the core of tissue from the body, thereby defining at least a portion of a tissue cavity;
analyzing the tissue core sample;
performing therapeutic management of tissue;
sealing the tissue cavity;
removing some or all instrumentation; and
closing tissue access points.

32. The method of claim 31, wherein the core of tissue comprises at least a portion of the tissue lesion.

33. The method of claim 31, wherein the determining the location of a tissue lesion comprises using an imaging system.

34. The method of claim 33, wherein the imaging system comprises one or more of computer tomography, ultrasound, magnetic resonance imaging, endoscope, visual, electromagnetic, or X-ray.

35. The method of claim 31, wherein the navigating an instrument to the tissue lesion comprising using a robotic system.

36. The method of claim 31, wherein the navigating an instrument to the tissue lesion comprising using one or more of computer tomography, ultrasound, electromagnetic navigation, or fluoroscopic navigation.

37. The method of claim 31, wherein the configuring access to the tissue site comprises using one or more of one or more of chest wall incision blades, tissue dilation, a trocar, or an open incision.

38. The method of claim 31, wherein the analyzing the tissue core sample comprises using one or more of rapid on-site evaluation, histology, immunohistochemistry, electron microscopy, flow cytometry, image cytometry, polymerase chain reaction, gene expression microarrays, fluorescent in situ hybridization, genetic sequencing, atomic force microscopy, or surface enhanced Ramen spectroscopy.

39. The method of claim 31, wherein the performing therapeutic management of tissue comprises one or more of ablation, chemotherapy, hydrogel-based controlled drug delivery, or photodynamic therapy.

40. The method of claim 31, wherein the performing therapeutic management of tissue comprises using a scaffold system.

41. The method of claim 31, wherein the performing therapeutic management of tissue comprises hyperthermia of cored tissue site.

42. The method of claim 31, wherein the performing therapeutic management of tissue is based on the analyzing the tissue core sample.

43. The method of claim 31, wherein the performing therapeutic management of tissue is implemented on benign tissue.

44. The method of claim 31, wherein the performing therapeutic management of tissue is implemented on malignant tissue.

45. A method for coring tissue, the method comprising:
introducing a tissue resection device to a tissue site;
using the tissue resection device to create a core of tissue;
removing at least a portion of the core of tissue from the body, thereby defining at
least a portion of a tissue cavity;
analyzing the tissue core sample; and
sealing the tissue cavity.

46. The method of claim 45, further comprising using an imaging system to determine the location of a tissue lesion within the tissue site.

47. The method of claim 46, wherein the imaging system comprises one or more of computer tomography, ultrasound, magnetic resonance imaging, endoscope, visual, electromagnetic, or X-ray.

48. The method of claim 45, further comprising using a robotic system to navigate an instrument to the tissue site.

49. The method of claim 45, further comprising using one or more of computer tomography, ultrasound, electromagnetic navigation, or fluoroscopic navigation to navigate an instrument to the tissue site.

50. The method of claim 45, wherein the analyzing the tissue core sample comprises using one or more of rapid on-site evaluation, histology, immunohistochemistry, electron microscopy, flow cytometry, image cytometry, polymerase chain reaction, gene expression microarrays, fluorescent in situ hybridization, genetic sequencing, atomic force microscopy, or surface enhanced Ramen spectroscopy.

51. The method of claim 45, further comprising determining a diagnostic using at least the tissue core sample.

52. The method of claim 45, further comprising performing therapeutic management of tissue.

53. The method of claim 45, further comprising performing therapeutic management of benign tissue.

54. The method of claim 45, further comprising performing therapeutic management of malignant tissue.

55. The method of claim 54, wherein the performing therapeutic management of malignant tissue comprises ablation.

56. The method of claim 54, wherein the performing therapeutic management of tissue comprises one or more of ablation, chemotherapy, hydrogel-based controlled drug delivery, or photodynamic therapy.

57. The method of claim 54, wherein the performing therapeutic management of malignant tissue comprises using a scaffold system.

58. The method of claim 54, wherein the performing therapeutic management of malignant tissue comprises hyperthermia of cored tissue site.

59. A method for coring tissue, the method comprising:
determining the location of a tissue lesion;
navigating an instrument based on a location of the tissue lesion;
coupling the instrument to the tissue lesion or adjacent the tissue lesion;
introducing a tissue resection device to a tissue site;
using the tissue resection device to create a core of tissue;
removing the core of tissue from the body, thereby defining at least a portion of a tissue cavity;
analyzing the tissue core sample;
performing therapeutic management of tissue;
sealing the tissue cavity; and
removing some or all instrumentation.

60. The method of claim 59, wherein the core of tissue comprises at least a portion of the tissue lesion.

61. The method of claim 59, wherein the determining the location of a tissue lesion comprises using an imaging system.

62. The method of claim 61, wherein the imaging system comprises one or more of computer tomography, ultrasound, magnetic resonance imaging, endoscope, visual, electromagnetic, or X-ray.

63. The method of claim 59, wherein the navigating an instrument to the tissue lesion comprising using a robotic system.

64. The method of claim 59, wherein the navigating an instrument to the tissue lesion comprising using one or more of computer tomography, ultrasound, electromagnetic navigation, or fluoroscopic navigation.

65. The method of claim 59, further comprising configuring access to the tissue site wherein the configuring access to the tissue site comprises using one or more of one or more of chest wall incision blades, tissue dilation, a trocar, or an open incision.

66. The method of claim 59, wherein the analyzing the tissue core sample comprises using one or more of rapid on-site evaluation, histology, immunohistochemistry, electron microscopy, flow cytometry, image cytometry, polymerase chain reaction, gene expression microarrays, fluorescent in situ hybridization, genetic sequencing, atomic force microscopy, or surface enhanced Ramen spectroscopy.

67. The method of claim 59, wherein the performing therapeutic management of tissue comprises one or more of ablation, chemotherapy, hydrogel-based controlled drug delivery, or photodynamic therapy.

68. The method of claim 59, wherein the performing therapeutic management of tissue comprises using a scaffold system.

69. The method of claim 59, wherein the performing therapeutic management of tissue comprises hyperthermia of cored tissue site.

70. The method of claim 59, wherein the performing therapeutic management of tissue is based on the analyzing the tissue core sample.

71. The method of claim 59, wherein the performing therapeutic management of tissue is implemented on benign tissue.

72. The method of claim 59, wherein the performing therapeutic management of tissue is implemented on malignant tissue.
